(19) United States
(12) Patent Application Publication
(10) Pub. No.: US 2009/0041820 A1
(43) Pub. Date: Feb. 12, 2009

(54) Functional polymer compositions

(76) Inventors: Margaret M. Wu, Skillman, NJ (US); Bruce R. Lundmark, Akron, OH (US); Bryan R. Chapman, Annandale, NJ (US); Hyun-Dae Jung, Singapore (SG); John Andres Harvey, Katy, TX (US); Chia Yung Cheng, Seabrook, TX (US); Anthony James Giaquinto, JR., Houston, TX (US); James Zielinski, Somerset, NJ (US); William Grant Britton, Katy, TX (US); David B. Dunaway, Houston, TX (US)

Correspondence Address: ExxonMobil Research and Engineering Company, P.O. Box 900, Annandale, NJ, 08801-0900, US

(21) Appl. No.: 12/221,278

(22) Filed: Aug. 1, 2008

Related U.S. Application Data
(60) Provisional application No. 60/963,799, filed on Aug. 7, 2007.

(51) Int. Cl. A01N 25/08; A61K 47/00; A61K 36/886; A61K 31/075; A61K 33/40; A61L 9/01; B29C 49/00; B05D 1/02; A61K 8/18; C11D 3/37; A61K 31/13; A61K 33/00; A61K 9/14

(52) U.S. Cl. 424/409; 514/772.3; 424/744; 514/721; 424/613; 514/662; 424/600; 424/489; 510/527; 523/122; 252/194; 512/8; 264/572; 427/427.6

(57) ABSTRACT

Functional polymer compositions and methods of forming such compositions are provided. The functional polymer compositions include at least 50 wt % of one or more polymer components; from 0.1 to 50 wt % of one or more fluids; and from 1 to 50 wt % of one or more active substrates. The combination of one or more fluids and one or more active substrates provides an article from such polymer composition with one or more special functional effects selected from moisturizing, anti-bacterial, disinfecting, anti-viral, anti-mildew, anti-mold, anti-fungal, anti-microbial, moisture/odor absorbing, fragrancing, insect repelling, anti-static and combinations thereof. The one or more fluids and one or more active substrates are released to the surface of polymer based articles. The functional polymer compositions are suitable for a wide range of end use applications including woven and non-woven fabrics.

### CROSS-REFERENCE TO RELATED APPLICATIONS

This is a Non-Provisional Application that claims priority to U.S. Provisional Application 60/963,799 filed Aug. 7, 2007, which is herein incorporated by reference.

### FIELD

The present disclosure is directed generally to functional polymer compositions. More particularly, it relates to functional polymer compositions and methods of producing such compositions that include a polymeric base substrate with one or more active substrates and one or more fluids, and the functional polymer composition imparts special functionality to polymer based products, such as films, fibers, fabrics, extruded products and molded products formed from such compositions.

### BACKGROUND

Many products are formed from polymeric compositions. Among these products are films, fibers, fabrics derived from fibers, extruded products and molded products. It is often desirable to improve the utility of such products by providing them with novel functionality. For example, polyolefins and other fiber forming polymers are commonly used as the base synthetic polymer for many synthetic non-woven or woven fabrics because of their advantageous properties, processability and cost, especially when the products are used in disposal or semi-disposal type applications, but also in some non-disposal type applications. Examples of these polymer-based non-woven and woven fabric products are underwear, gloves, socks, face masks, hair cover, protective clothing, sports clothing and wipes. These non-woven and woven fabrics are used in general for personal care, hospital care, institutional care, patient care, baby care, adult incontinence care, travel, and sporting events. Fibers formed from synthetic polymer compositions are also used to produce carpets for industrial and residential uses. Existing commercial products perform the basic function with minimum concern for additional comfort, aesthetics, or the ability to deliver extra benefits, such as moisturizing, anti-septic, fragrance, anti-fungal, anti-bacterial or anti-viral, anti-microbial effect, sweat-absorbing, odor mitigation, or other special functionality.

U.S. Pat. No. 6,626,961 discloses a non-woven fabric treated with an aqueous-based petrolatum-surfactant mixture, which imparts skin health benefits during product use. The petrolatum-surfactant mixture can be applied to the non-woven fabric by a foaming process. The surface-modified non-woven fabric comprises: a nonwoven web treated with an aqueous-based dispersion of a petrolatum-surfactant mixture, which includes between 60 wt % and 99 wt % water. The petrolatum surfactant mixture includes petrolatum, a long chain fatty alcohol, an ethoxylated fatty acid ester of a sugar, and a monofatty acid ester of a sugar.

PCT patent application No. WO199744011 A1 discloses a non-woven membrane system for controlled release of a soluble substance comprising thermoplastic polymer filaments and/or fibers with controlled diffusion properties, i.e. through which can diffuse in controlled manner a substance solution in a liquid such as water or an aqueous medium, when a solution of this substance in the liquid is in contact with one of the surfaces of the membrane, the other surface being in contact with the liquid.

PCT patent application No. WO2004014998A2 discloses a plasticized polyolefin composition comprising one or more polyolefins and one or more non-functionalized plasticizers. U.S. Patent application No. 20040186214 discloses fibers and non-wovens made from plasticized polyolefin compositions comprising a polyolefin and a non-functionalized hydrocarbon plasticizer. US Patent application No. 2006008643 discloses fibers and non-wovens made from plasticized polyolefin compositions comprising a polyolefin, a non-functionalized hydrocarbon plasticizer, and a slip agent. In each of these cases, the non-functionalized plasticizer is a compound comprising carbon and hydrogen, and does not include to an appreciable extent functional groups selected from hydroxide, aryls and substituted aryls, halogens, alkoxys, carboxylates, esters, carbon unsaturation, acrylates, oxygen, nitrogen, and carboxyl.

PCT patent application WO200230194A1 and US Patent application 2004063806 disclose the use of polyalphaolefin fluids as carriers for antimicrobial agents into polyolefins.

In order to meet customers' desire for more comfort, better feel and added value, it is desirable to have polymer compositions used to produce films, fibers, fabrics, extruded products and molded products perform not only their basic duty, but also impart extra functionality. Hence a need exists for improved polymer compositions and systems that impart additional functionality for their intended end-use application as well as methods to produce such polymer compositions and methods to form such compositions into finished films, fibers, fabrics, extruded products and molded products for consumers' needs. A need also exists for polymer compositions and systems that allow for controlled migration of functional components contained within the composition.

### SUMMARY

Functional polymer compositions are provided herein. These functional compositions contain one or more base polymers, one or more functional fluids and/or one or more active. The compositions are formulated in a manner to allow controlled release of the functional fluid(s) and/or the active agent(s) during use.

According to the present disclosure, an advantageous functional polymer composition comprises: (a) at least 50 wt %, based on the total weight of the composition, of one or more thermoplastic polymers; (b) from 0.1 to 50 wt %, based on the total weight of the composition, of one or more fluids; (c) from 0.1 to 50 wt %, based on the total weight of the composition, of one or more active substrates; and wherein an article formed from the composition exhibits one or more special functional effects from controlled release to the article surface of the one or more fluids and one or more active substrates.

Another aspect of the present disclosure relates to an advantageous functional polymer composition comprising: (a) at least 50 wt %, based on the total weight of the composition, of one or more thermoplastic polymers; and (b) from 0.1 to 50 wt %, based on the total weight of the composition, of one or more fluids; wherein an article formed from the composition exhibits one or more special functional effects from controlled-release to the article surface of the one or more fluids.

Still another aspect of the present disclosure relates to an advantageous functional polymer composition comprising: (a) at least 50 wt %, based on the total weight of the composition, of one or more thermoplastic polymers; (b) from 0.1 to 50 wt %, based on the total weight of the composition, of one or more active substrates; and wherein an article formed from the composition exhibits one or more special functional effects from controlled release of the one or more active substrates to the article surface.

Still yet another aspect of the present disclosure relates to an advantageous functional polymer composition comprising: (a) at least 50 wt %, based on the total weight of the composition, of one or more thermoplastic polymers; (b) from 0.1 to 50 wt %, based on the total weight of the composition, of a fluid mixture of PAO and ester fluids, wherein the fluid mixture comprises 50 to 99 wt % of PAO fluid and 1 to 50 wt % of ester fluid, based on the total weight of the fluid mixture; (c) from 0.1 to 30 wt %, based on the total weight of the composition, of triclosan; and wherein an article formed from the composition exhibits an anti-bacterial effect from migration to the article surface of the fluid mixture and the triclosan.

Still yet another aspect of the present disclosure relates to an advantageous method for producing functional polymer compositions comprising: providing a composition comprising (a) at least 50 wt %, based on the total weight of the composition, of one or more thermoplastic polymers; (b) from 0.0 to 50 wt %, based on the total weight of the composition, of one or more fluids; (c) from 0.1 to 50 wt %, based on the total weight of the composition, of one or more active substrates agents; and mixing the composition, and forming the composition into an article, wherein the article exhibits one or more special functional effects from controlled release to the article surface of the one or more fluids and one or more active substrates.

Still yet another aspect of the present disclosure relates to an advantageous method for producing functional polymer compositions comprising: providing a fluid composition comprising: (a) from 0.0 to 95 wt %, based on the total weight of the composition, of one or more fluids; (b) from 0.1 to 95 wt %, based on the total weight of the composition, of one or more active substrates; and mixing the fluid composition; and spraying or impregnating the fluid composition onto the surface of a polymer based article, wherein the article exhibits one or more special functional effects from migration to the article surface of the one or more fluids and one or more active substrates.

These and other features and attributes of the disclosed functional polymer compositions and methods for producing such compositions of the present disclosure and their advantageous applications and/or uses will be apparent from the detailed description which follows.

### DETAILED DESCRIPTION

The present disclosure relates to improved functional polymer compositions, methods of producing such compositions and uses of such compositions to form films, fibers, and/or fabrics, including woven, non-woven fabrics, fiber-derived articles (such as ropes, strings, patches), and carpets and rugs, extruded products and molded products. The functional polymer compositions of the present disclosure include a combination of one or more thermoplastic polymer(s), one or more fluids, and/or one or more active substrates that impart improved functionality to the polymer compositions and products produced there from.

The functional polymer compositions are distinguishable over the prior art by incorporating one or more active substrates agent(s) and/or fluids which impart special functions/attributes by way of their transport of the active agent(s) to the polymer matrix surface at a controlled rate. Furthermore, one or more carrier agent(s) may be employed to synergistically promote the function of the active substrate agent(s) by facilitating their transport to the polymer matrix surface at a controlled rate. Such a system allows for the controlled migration and/or controlled release of the one or more modifying fluid(s) and/or active agent(s) to the polymer matrix surface.

The term “polymer matrix” as defined herein is used to describe the solid articles made from the polymeric compositions; it refers to the solid film, fiber, molded products, fabric, etc. The term “fluid” as defined herein is used to describe a liquid component in the polymer system. The term “active agent” as defined herein is used to describe a component which, when added to the system, imparts a special functionality not typically associated with similar articles made from the base polymer(s). All numerical values within the detailed description and the claims herein are understood as modified by “about.”

### Polymer Components:

Polyolefins and other thermoplastic polymers are used to produce the base material for the functional polymer compositions disclosed herein. Exemplary thermoplastic polymers include, but are not limited to, one or more of the following: polyolefins, polystyrenes, polyesters, polyamides, synthetic rubber, natural rubber, and combinations thereof. Examples of polyolefins include polyethylene, polypropylene, polybutene, copolymers of ethylene and propylene (EP copolymer), copolymers of ethylene and butene-1 (EB copolymer), copolymers of ethylene and hexene-1, copolymers of ethylene and octene-1, copolymers of propylene and butene-1 (PB copolymer), terpolymers of ethylene, propylene and butene-1 (EPB terpolymer), ethylene and/or propylene copolymers with one or more C₄ to C₂₀ alpha-olefins, copolymers of ethylene and vinyl acetate (EVA copolymer), ethylene plastomer, synthetic rubber, blends of synthetic and natural rubber, maleic anhydride modified polyethylene or polypropylene homopolymers or copolymers, ethylene-alpha-olefin elastomers (EE), polyolefin adhesive (POA), styrene-polyethylene-alpha-olefin elastomers, ethylene elastomer, styrene-diene copolymer, butyl rubber. (polyisobutylene rubber). Examples of polystyrenes include polystyrene and copolymers of styrene with one or more C₂ to C₂₀ olefins and/or diolefins. Examples of polyesters include polyethylene terephalate (PET) and polybutylene terephalate (PBT). Examples of polyamides include Nylon 6, Nylon 6,6, Nylon 4,6, and Nylon 6,12 and combinations of any of the preceding polymer types. Examples of synthetic rubbers include terpolymers of ethylene/propylene/diene (EPDM), butyl rubber, polyisoprene, polybutadiene, polyisobutylene, and styrene/butadiene rubber (SBR). Generally, these one or more base polymers may be formed into films, fibers, or fiber-derived articles such as woven or non-woven fabrics (woven or non-woven), other extruded product forms and/or a variety of molded product forms (injection molding, compression molding, thermoforming, etc.).

International Patent Publication No. WO 2006083540 discloses modified polyethylene compositions and pages 33 to 41 of such publication are herein incorporated by reference relative to the range of ethylene based polymer that may be used for the base polymer of this disclosure. International Patent Publication No. WO 2004014998 discloses plasticized polyolefin compositions and pages 29 to 53 of such publication are herein incorporated by reference relative to the range of propylene based polymers and butene based polymers that may be used for the base polymer of this disclosure.

The base polymeric substrate, which includes one or more of the aforementioned polymer types, of the functional polymer composition generally comprises at least 50 wt %, or at least 60 wt %, or at least 70 wt %, or at least 80 wt %, or at least 90 wt %, or at least 95 wt %, or at least 96 wt %, or at least 97 wt %, or at least 98 wt %, or at least 99 wt % of the functional polymer composition.

The one or more synthetic base polymers may include one or more natural fibers or blends of natural fibers as additives. Exemplary, but not limiting, natural fibers that may be combined with the synthetic fibers include cotton, wool, silk, and combinations thereof. Generally, natural fibers are incorporated into the base fabric at less than 50 wt %, or less than 40 wt %, or less than 30 wt %, or less than 20 wt %, or less than 10 wt %.

In addition to the one or more synthetic base polymers, the polymer compositions disclosed herein may also include one or more other additives, including, but not limited to, fillers, cavitating agents, antioxidants, surfactants, adjuvants, plasticizers, block, antiblock, coloring agents, pigments, dyes, processing aids, UV stabilizers, neutralizers, waxes, nucleating agents, foaming agents, reinforcing fibers, antistatic agents, lubricating agents, and/or clarifying agents. These and other polymer additives are described in further detail on pages 41 to 47 of in International Patent Publication No. WO 2006/083540, which is herein incorporated by reference. These polymer additives may be added to the one or more base polymers at ratios of up to 40 wt %, or up to 30 wt %, or up to 20 wt %, or up to 10 wt %, or up to 5 wt %, or up to 1 wt % to further improve product quality and product properties. In another embodiment, these additives may be present in the amounts from 0.001 wt % to 10 wt %, or 0.05 to 5 wt %, or 0.01 to 1 wt %.

Further details of the base polymers, the polymer blends, and the polymer additives for use with the functional polymer compositions disclosed herein may be found on pages 53-71 of International Patent Publication No. WO2004014998, herein incorporated by reference.

### Fluid Components:

Unless otherwise specified, all fluid viscosities disclosed herein are the 100° C. kinematic viscosity in cSt (or centi-Stoke), measured according to ASTM D445 method. One or more fluid components may also be included in the functional polymer compositions to impart special functionality and/or to assist with transport of one or more active substrates to the polymer matrix surface. In one form, the one or more fluids may be utilized with or without one or more active substrates to impart special functionality to the functional polymer matrix. In another form, one or more fluids may be used to assist with controlled migration of the one or more active substrates within the functional polymer matrix. In particular, the one or more fluids may act as carriers for the transport of the one or more active substrates in the functional polymer matrix to permit time release migration of the active substrates from the bulk of the polymer composition to the surface of the functional polymer matrix. This time released aspect permits the functionality to be sustained within the functional polymer matrix for extended periods of time. In yet another form, one or more fluids may be used to both impart special functionality to the functional polymer matrix and also to provide for controlled migration or release of the fluid and/or the active substrates from the bulk of the matrix to the surface of the matrix. In yet another aspect, the presence of this fluid can facilitate the controlled release of the other normally non-migratory substrates in the polymer matrix onto the surface or closer to the surface to perform its function.

For many active substrates, such as an anti-microbial (AM) agent, it is most effective to have the AM agent at the surface of the article or to have a consistent high concentration of the AM agent at the surface of the article, where the microbial species most like to reside. In this manner, the AM will have highest effectiveness. Or in some cases, if the article is used by a person, such as a glove, face mask or socks, etc., a steady, but small and controlled amount of the AM will be slowly rubbed off from the surface of the article to the user to offer effective anti-microbial effect.

Many factors impact the most effective controlled release of the functional fluids and the active substrates. This is a very complex problem. Generally, the choice of the base polymer compositions affects the release rates of fluids in combination with the active functional substrates. Different polymer classes, such as polyethylene, polypropylene, polyesters, polybutenes, ethylene-propylene elastomers, etc., have different degrees of crystallinity, void volume, compatibility, polymer internal pressures, solubility, etc. These properties effect the migration and release of the fluids and active components. For a given class of polymer, the molecular weight, molecular weight distribution, polymer density, degree of crystallinity, crystallite size and shape, method of fabrication, etc. may have an effect on the degree and the rate of release or migration of the fluids and/or active substrate components.

Unless otherwise specified, all fluid viscosities disclosed herein are the 100° C. kinematic viscosity in cSt (or centi-Stoke), measured according to ASTM D445 method. Exemplary fluids include, but are not limited to, one or more of the following: low molecular weight oligomers or polymers of olefins, paraffinic hydrocarbons (such as certain types of lubricant basestocks and paraffinic mineral oils), ester fluids, polyester fluids, alkylated aromatic fluids, polyalkylene glycols, and silicone fluids. These fluids generally have kinematic viscosities at 40° C. of greater than 2 cSt. Other non-limiting examples of fluids include hydrocarbon solvents, poly-alpha-olefins (PAO), polyisobutylene (PIB), esters of mono-, di- or tri-basic acids, esters of acids with mono-, di- or polyols, EO/PO copolymers, polypropylene oxides, poly-butyleneoxides, polytetrahydrofuran ether/ester/alcohol, alkylated naphthalene, alkylated aromatics, dimethicone, cyclomethicone and other polysiloxane or silicone fluids, paraffinic mineral oils, lubricant basestock derived from iso paraffin-rich liquid derived from gas-to-liquid processes, and other fluids described in SYNTHETICS, MINERAL OILS AND BIO-BASED LUBRICANTS, CHEMISTRY AND TECHNOLOGY, ed. by L. R. Rudnick, published in 2006 by CRC Press, Taylor & Francis Group, Boca Raton, Fla. Mixtures of one or more of the above fluids may also be advantageous. Some of these fluid modifiers are described on pages 12 to 33 of International Patent Publication No. WO 2006/083540, herein incorporated by reference.

Such fluids may provide unique properties, independent or synergistic to the properties attributable to the active agent(s), such as a moisturizing effect or a skin surface film with improved barrier properties to prevent skin moisture evaporation. Alternatively, these fluids may act as a carrier agent to help deliver other active substrates. The one or more fluid components are generally incorporated into the functional polymer composition in the range of 0.1 to 50 wt %, or from 0.3 to 30 wt %, or from 0.5 to 25 wt %, or from 1 to 20 wt %. The lower limit for the one or more fluid components in the functional polymer composition may be 0.1, or 0.2, or 0.3, or 0.4, or 0.5, or 1.0, or 2.0 or 3.0, or 4.0, or 5.0, or 7.5, or 10, or 15, or 20 wt %. The upper limit for the one or more fluid components in the functional polymer composition may be 10, or 15, or 20, or 25, or 30, or 35, or 40, or 45, or 50 wt %. The amount to be incorporated depends on the function and the application. Particularly advantageous fluids and blends of fluids are those having a) a flash point of 150° C. or more, advantageously 200° C. or more, or 210° C. or more, or 220° C. or more, or 230° C. or more; and b) a pour point less than 0° C., advantageously less than −25° C., or less than −30° C., or less than −35° C., or less than −40° C.) and/or a KV100° C. of 4 cSt or more, advantageously 35 cSt or more, or 40 cSt or more, or 60 cSt or more.

Further advantageous modifying fluids and blends of modifying fluids have a KV100° C. of at least 2 cSt, advantageously at least 3 cSt, or at least 4 cSt, or at least 6 cSt, or at least 8 cSt, or at least 10 cSt; a VI of at least 120, advantageously at least 130, or at least 140, or at least 150; a pour point of −10° C. or less, advantageously −20° C. or less, or −30° C. or less, or −40° C. or less.

a. Polyalphaolefin (PAO) Fluids:

Polyalphaolefins (PAOs) are a particularly advantageous class of fluids for use in the present disclosure. In one form, the polyalphaolefin fluid has a pour point of −10° C. or less and a kinematic viscosity at 100° C. (KV100° C.) of 2 cSt or more. PAO liquids are described in, for example, U.S. Pat. Nos. 3,149,178; 4,827,064; 4,827,073; 5,171,908; 5,783,531, International Patent Publication Nos. WO2007011832 A1, and WO2007011459 A1, and in SYNTHETIC LUBRICANTS AND HIGH-PERFORMANCE FUNCTIONAL FLUIDS, (Leslie R. Rudnick & Ronald L. Shubkin, ed. Marcel Dekker, Inc. 1999), p. 3-52.

PAOs are high purity hydrocarbons with a fully paraffinic structure. PAO liquids can be conveniently prepared by the oligomerization of an alpha-olefin in the presence of a polymerization catalyst, such as a Friedel-Crafts catalyst including BF₃ or AlCl₃ or their promoted analogs, coordination complex catalyst (including, for example, the ethylaluminum sesquichloride+TiCl₄ system), or a homogeneous or heterogeneous (supported) catalyst more commonly used to make polyethylene and/or polypropylene (including, for example, Ziegler-Natta catalysts, metallocene or other single-site catalysts, and chromium catalysts).

In one or more forms, the PAO comprises C₁₅ to C₁₅₀₀ or C₂₀ to C₁₀₀₀, or C₃₀ to C₈₀₀, or C₃₅ to C₄₀₀, or C₄₀ to C₂₅₀ oligomers of C₃ to C₂₄, or C₅ to C₁₈, or C₆ to C₁₄, or C₈ to C₁₂ alpha-olefins. The use of linear alpha-olefins (LAOs) is advantageous and the use of C₈ to C₁₂, are particularly advantageous, provided that C₃ and C₄ alpha-olefins are present at 30 wt % or less, or 20 wt % or less, or 10 wt % or less, or 5 wt % or less. Suitable LAOs include propylene, 1-butene, 1-pentene, 1-hexene, 1-heptene, 1-octene, 1-nonene, 1-decene, 1-undecene, 1-dodecene, 1-tridecene, 1-tetradecene, 1-pentadecene, 1-hexadecene, and blends thereof.

In one or more forms, a single LAO is used to prepare the oligomers. The oligomerization of 1-octene is advantageous and the oligomerization of 1-decene particularly advantageous.

In one or more forms, the PAO comprises oligomers of two or more C₃ to C₁₈ LAOs, to make ‘bipolymer’ or ‘terpolymer’ or higher-order copolymer combinations, provided that C₃ and C₄ LAOs are present 30 wt % or less (advantageously 20 wt % or less, advantageously 10 wt % or less, advantageously 5 wt % or less). An advantageous form involves oligomerization of a mixture of LAOs selected from C₆ to C₁₈ LAOs with even carbon numbers. Another advantageous form involves oligomerization of a mixture of LAOs selected from 1-octene, 1-decene, and 1-dodecene.

In one or more forms, the PAO comprises oligomers of a single alpha-olefin species having a carbon number of 5 to 24, or 6 to 18, or 8 to 12, or 10. Oligomers of a single alpha-olefin species having a carbon number of 10 are advantageous. In one or more forms, the PAOs comprise oligomers of mixed alpha-olefins (i.e., two or more alpha-olefin species), each alpha-olefin having a carbon number of 5 to 24, or 6 to 18, or 8 to 12. In one or more forms, the PAO comprises oligomers of mixed alpha-olefins (i.e., involving two or more alpha-olefin species) where the weighted average carbon number for the alpha-olefin mixture is 6 to 14, or 8 to 12, or 9 to 11. Mixed alpha-olefins with weighted average carbon number for the alpha-olefin mixture of 8 to 12, or 9 to 11 are advantageous, and with weighted average carbon number for the alpha-olefin mixture of 9 to 11 are particularly advantageous.

In one or more forms, the PAO comprises oligomers of one or more alpha-olefin with repeat unit formulas of —[CHR—CH₂]-where R is a C₃ to C₁₈ saturated hydrocarbon branch. In one or more forms, R is constant for all oligomers. In one or more forms, there is a range of R substituents covering carbon numbers from 3 to 18. Advantageously, R is linear, i.e., R is (CH₂)ₙCH₃, where n is 3 to 17, or 4 to 11, and or 5 to 9. Linear R groups with n of 4 to 11, and or 5 to 9 are advantageous, and with n of 5 to 9 are particularly advantageous. Optionally, R can contain one methyl or ethyl branch, i.e., R is (CH₂)ₘ[CH(CH₃)](CH₂)_{z}CH₃ or R is (CH₂)ₓ[CH(CH₂CH₃)](CH₂)_{y}CH₃, where (m+z) is 1 to 15, advantageously 1 to 9, or 3 to 7, and (x+y) is 1 to 14, advantageously 1 to 8, or 2 to 6. Advantageously m>z; m is 0 to 15, advantageously 2 to 15, or 3 to 12, more advantageously 4 to 9; and n is 0 to 10, advantageously 1 to 8, or 1 to 6, more advantageously 1 to 4. Advantageously x>y; x is 0 to 14, advantageously 1 to 14, or 2 to 11, more advantageously 3 to 8; and y is 0 to 10, advantageously 1 to 8, or 1 to 6, more advantageously 1 to 4. Advantageously, the repeat units are arranged in a head-to-tail fashion with minimal head-to-head connections.

The PAO can be atactic, isotactic, or syndiotactic. In one or more forms, the PAO has essentially the same population and random distribution of meso and racemic diads, on average, making it atactic. In one or more forms, the PAO has more than 50%, or more than 60%, or more than 70%, or more than 80%, advantageously more than 90% meso dyads (i.e., [mm]) as measured by ¹³C-NMR. In one or more forms, the PAO has more than 50%, or more than 60%, or more than 70%, or more than 80%, advantageously more than 90% racemic diads (i.e., [rr]) as measured by ¹³C-NMR.

The PAO liquid can include one or more distinct PAO components. In one or more form, the fluid is a blend of one or more PAOs with different compositions and/or different physical properties (e.g., kinematic viscosity, pour point, and/or viscosity index).

In one or more forms, the PAO or blend of PAOs has a number-averaged molecular weight (Mₙ) of from 400 to 15,000 g/mol, advantageously 400 to 12,000 g/mol, or 500 to 10,000 g/mol, or 600 to 8,000 g/mol, more advantageously 800 to 6,000 g/mol, or 1,000 to 5,000 g/mol). In one or more forms, the PAO or blend of PAOs has a Mₙ greater than 1,000 g/mol, or greater than 1,500 g/mol, advantageously greater than 2,000 g/mol, or greater than 2,500 g/mol).

In one or more forms, the PAO or blend of PAOs has a KV100° C. of 2 cSt or more, 3 cSt or more, or 4 cSt or more, or 5 cSt or more, or 6 cSt or more, or 8 cSt or more, or 10 cSt or more, or 20 cSt or more, or 30 cSt or more, or 40 cSt or more, advantageously 100 or more, or 150 cSt or more. In one or more forms, the PAO has a KV100° C. of 3 to 3,000 cSt, or 4 to 1,000 cSt, advantageously 6 to 300 cSt, or 8 to 150 cSt, or 8 to 100 cSt, or 8 to 40 cSt). In one or more forms, the PAO or blend of PAOs has a KV100° C. of 10 to 1000 cSt, advantageously 10 to 300 cSt, or 10 to 100 cSt. In yet another form, the PAO or blend of PAOs has a KV100° C. of 4 to 8 cSt. In yet another form, the PAO or blend of PAOs has a KV100° C. of 25 to 300 cSt, advantageously 40 to 300 cSt, or 40 to 150 cSt. In one or more forms, the PAO or blend of PAOs has a KV100° C. of 100 to 300 cSt.

In one or more forms, the PAO or blend of PAOs has a Viscosity Index (VI) of 100 or more, of 120 or more, advantageously 130 or more, or 140 or more, or 150 or more, or 170 or more, or 190 or more, or 200 or more, advantageously 250 or more, or 300 or more. In one or more forms, the PAO or blend of PAOs has a VI of 120 to 350, advantageously 130 to 250.

In one or more forms, the PAO or blend of PAOs has a pour point of −10° C. or less, advantageously −20° C. or less, or −25° C. or less, or −30° C. or less, or −35° C. or less, or −40° C. or less, or −50° C. or less. In one or more forms, the PAO or blend of PAOs has a pour point of −15 to −70° C., advantageously −25 to −60° C.

In one or more forms, the PAO or blend of PAOs has a glass transition temperature (T_{g}) of −40° C. or less, advantageously −50° C. or less, or −60° C. or less, or −70° C. or less, or −80° C. or less. In one or more forms, the PAO or blend of PAOs has a glass transition temperature (T_{g}) measured by differential thermal calorimetry of −50 to −120° C., advantageously −60 to −100° C., or −70 to −90° C.

In one or more forms, the PAO or blend of PAOs has a flash point of 150° C. or more, of 175° C. or more, of 200° C. or more, advantageously 210° C. or more, or 220° C. or more, or 230° C. or more, more advantageously between 240° C. and 290° C.

In one or more forms, the PAO or blend of PAOs has a specific gravity (15.6° C.) of 0.89 or less, 0.86 or less, advantageously 0.855 or less, or 0.85 or less, or 0.84 or less.

In one or more forms, the PAO made directly from the production process may be used. In this case, the PAO may have a molecular weight, as defined by the ratio of Mw/Mn, of less than 4, or less than 3.5, or less than 3.0, or less than 2.5, or less than 1.5, or less than 1.2, or less than 1.1. Often the blends of these directly produced PAOs may be used. The PAO blends may be prepared by blending very low viscosity fluids with very high viscosity fluids, or by blending two fluids of comparable viscosities. The PAO blends have a molecular weight distribution as characterized by the ratio of the weight- and number-averaged molecular weights (M_{w}/Mₙ) of 1.5 or more, advantageously 2 or more, advantageously 2.5 or more, or 3 or more, or 4 or more, or 5 or more, or 6 or more, or 8 or more, or 10 or more. In one or more embodiments, the PAO or blend of PAOs has a M_{w}/Mₙ of 5 or less, advantageously 4 or less, or 3 or less and a KV1100° C. of 10 cSt or more, advantageously 20 cSt or more, or 40 cSt or more, or 60 cSt or more.

In one or more forms, the PAO or blend of PAOs has a Noack volatility of less than N* where N*=60e^{-0.4(KV100° C.)} with N* in units of weight % and KV100° C. of the fluid in question in units of cSt. In an additional form, KV100° C. of the fluid in question is less than 1000 cSt, advantageously less than 300 cSt, or less than 150 cSt, or less than 100 cSt, or less than 40 cSt, or less than 25 cSt, or less than 10 cSt, or less than 8 cSt.

Advantageous blends of PAOs include blends of two or more PAOs where the ratio of the highest KV100° C. to the lowest KV100° C. is at least 1.5, advantageously at least 2, or at least 3, or at least 5. Also advantageous are blends of two or more PAOs wherein at least one PAO has a Noack volatility of less than N* as defined above; advantageously all the PAOs in a blend have a Noack volatility of less than N*. In an additional embodiment, KV100° C. of the PAOs are less than 1000 cSt, advantageously less than 300 cSt, or less than 150 cSt, or less than 100 cSt, or less than 40 cSt, or less than 25 cSt, or less than 10 cSt, or less than 8 cSt.

Advantageous blends of PAO also include: blends of two or more PAOs where at least one PAO has a KV100° C. of 300 cSt or more and at least one PAO has a KV100° C. of less than 300 cSt, advantageously 150 cSt or less, or 100 cSt or less, or 40 cSt or less; blends of two or more PAOs where at least one PAO has a KV100° C. of 150 cSt or more and at least one PAO has a KV100° C. of less than 150 cSt, advantageously 100 cSt or less, or 40 cSt or less); blends of two or more PAOs where at least one PAO has a KV100° C. of 100 cSt or more and at least one PAO has a KV100° C. of less than 100 cSt, advantageously 40 cSt or less, or 25 cSt or less, or 10 cSt or less; blends of two or more PAOs where at least one PAO has a KV100° C. of 40 cSt or more and at least one PAO has a KV100° C. of less than 40 cSt, advantageously 25 cSt or less, or 10 cSt or less, or 8 cSt or less; blends of two or more PAOs where at least one PAO has a KV100° C. of 10 cSt or more and at least one PAO has a KV100° C. of less than 10 cSt, advantageously 8 cSt or less, or 6 cSt or less, or 4 cSt or less); blends of two or more PAOs where at least one PAO has a KV100° C. of 8 cSt or more and at least one PAO has a KV100° C. of less than 8 cSt, advantageously 6 cSt or less, or 4 cSt or less); and blends of two or more PAOs where at least one PAO has a KV100° C. of 6 cSt or more and at least one PAO has a KV100° C. of less than 6 cSt, advantageously 4 cSt or less).

Examples for PAO fluid components with desirable properties include commercial products, such as SpectraSyn™ and SpectraSyn Ultra™ from ExxonMobil Chemical (USA), some of which are summarized in Table 1 below. Other useful PAOs include those available as Synfluid™ from ChevronPhillips Chemical (USA), as Durasyn™ from Ineos (UK), as Nexbase™ from Neste Oil (Finland), and as Synton™ from Chemtura (USA).

TABLE 1
SpectraSyn ™ Series Polyalphaolefins:

| | KV40° C., | KV100° C., | Mₙ, | | Pour Point, | Specific | Flash | |
|---|---|---|---|---|---|---|---|---|
| | cSt | cSt | g/mol | VI | ° C. | gravity | Point, ° C. | Noack, wt % |
| SpectraSyn 4 | 19 | 4 | 450 | 126 | −66 | 0.820 | 220 | 14 |
| SpectraSyn | 17 | 4 | 440 | 126 | −60 | 0.820 | 228 | 12 |
| Plus 4 | | | | | | | | |
| SpectraSyn 6 | 31 | 6 | 540 | 138 | −57 | 0.827 | 246 | 6.4 |
| SpectraSyn | 30 | 6 | 560 | 143 | −54 | 0.827 | 246 | 5.0 |
| Plus 6 | | | | | | | | |
| SpectraSyn 8 | 48 | 8 | 640 | 139 | −48 | 0.833 | 260 | 4.1 |
| SpectraSyn | 66 | 10 | 720 | 137 | −48 | 0.835 | 266 | 3.2 |
| 10 | | | | | | | | |
| SpectraSyn | 396 | 39 | 1,700 | 147 | −36 | 0.850 | 281 | 1.6 |
| 40 | | | | | | | | |
| SpectraSyn | 1240 | 100 | 2,800 | 170 | −30 | 0.853 | 283 | 1.1 |
| 100 | | | | | | | | |
| SpectraSyn | 1,500 | 150 | 3,700 | 218 | −33 | 0.850 | >265 | 0.4 |
| Ultra 150 | | | | | | | | |
| SpectraSyn | 3,100 | 300 | 4,900 | 241 | −27 | 0.852 | >265 | 0.3 |
| Ultra 300 | | | | | | | | |
| SpectraSyn | 10,000 | 1,000 | 11,000 | 307 | −18 | 0.855 | >265 | 0.3 |
| Ultra 1000 | | | | | | | | |

KV40° C. = kinematic viscosity at 40° C.
KV100° C. = kinematic viscosity at 100° C.

b. Polybutenes:

In some forms of the present disclosure, the modifying fluid comprises oligomers of C₄ olefins (including 1-butene, 2-butene, isobutylene, and butadiene, and mixtures thereof) and up to 10 wt % other olefins, often referred to as a “polybutenes” liquid when the oligomers comprise primarily isobutylene and 1-butene. As used herein, the term “polybutenes” also includes homopolymer oligomers of isobutylene or 1-butene, copolymer oligomers of a C₄ raffinate stream, and copolymer oligomers of C₄ olefins with ethylene and/or propylene and/or C₅ olefins. Such liquids are commonly used as additives for polyolefins; e.g. to introduce tack or as a processing aid. The ratio of C₄ olefin isomers can vary by manufacturer and by grade, and the material may or may not be hydrogenated after synthesis. Polybutenes are described in, for example, SYNTHETIC LUBRICANTS AND HIGH-PERFORMANCE FUNCTIONAL FLUIDS (Leslie R. Rudnick & Ronald L. Shubkin, ed., Marcel Dekker 1999), p. 357-392.

Particularly advantageous polybutenes include those in which isobutylene derived units comprise 40 to 100 wt % (advantageously 40 to 99 wt %, advantageously 40 to 96 wt %) of the polymer; and/or the 1-butene derived units comprise 0 to 40 wt % (advantageously 2 to 40 wt %) of the copolymer; and/or the 2-butene derived units comprise 0 to 40 wt % (advantageously 0 to 30 wt %, advantageously 2 to 20 wt %) of the polymer. Suitable polybutenes may have a kinematic viscosity at 100° C. of 3 to 50,000 cSt (more commonly 5 to 3000 cSt), a pour point of −60 to 10° C. (more commonly −40 to 0° C.), and a number-average molecular weight of 300 to 10,000 g/mol (more commonly 500 to 2,000 g/mol).

Desirable polybutenes liquids are commercially available from a variety of sources including Innovene (Indopol™ grades) and Infineum (C-Series grades). When the C₄ olefin is exclusively isobutylene, the material is referred to as “polyisobutylene” or PIB. Commercial sources of PIB include Texas Petrochemical (TPC Enhanced PIB grades).

Commercially available Oligomers of C₄ olefin(s)

| | KV @ | | Pour | | Flash |
|---|---|---|---|---|---|
| | 100° C., | | Point, | Specific | Point, |
| Grade | cSt | VI | ° C. | gravity | ° C. |
| TPC 137 (PIB) | 6 | 132 | −51 | 0.843 | 120 |
| TPC 1105 (PIB) | 220 | 145 | −6 | 0.893 | 200 |
| TPC 1160 (PIB) | 660 | 190 | +3 | 0.903 | 230 |
| Innovene Indopol H-25 | 52 | 87 | −23 | 0.869 | ~150 |
| Innovene Indopol H-50 | 108 | 90 | −13 | 0.884 | ~190 |
| Innovene Indopol H-100 | 218 | 121 | −7 | 0.893 | ~210 |
| Infineum C9945 | 11 | ~75 | −34 | 0.854 | 170 |
| Infineum C9907 | 78 | ~100 | −15 | 0.878 | 204 |
| Infineum C9995 | 230 | ~130 | −7 | 0.888 | 212 |
| Infineum C9913 | 630 | ~175 | +10 | 0.888 | 240 |

c. Isoparaffin-Rich Liquids:

In another form of the present disclosure, the modifying fluid is an isoparaffin-rich liquid with a pour point of −50° C. or less (advantageously −60° C. or less) and a specific gravity of 0.84 or less (advantageously 0.83 or less). By “isoparaffin-rich” is meant that the modifying fluid comprises at least 50 wt % (advantageously at least 60 wt %, advantageously at least 70 wt %, advantageously at least 80 wt %, advantageously at least 90 wt %, advantageously 100 wt %) of C₆ to C₁₅₀ (advantageously C₆ to C₁₀₀, advantageously C₆ to C₂₅, advantageously C₈ to C₂₀) isoparaffins. Advantageously the paraffin chains possess C₁ to C₁₀ alkyl branching along at least a portion of each paraffin chain, where any combination of regio and stereo placement of the alkyl branches may be involved. Isoparaffin liquids may also include a minor amount of (less than 50 wt %, advantageously less than 30 wt %, advantageously less than 10 wt %) of cycloparaffins with isoparaffinic branched side chains.

In one embodiment, the number-average molecular weight of the isoparaffin-rich liquid is in the range of 100 to 1000 (advantageously 120 to 500, advantageously 150 to 300) g/mol. In another embodiment, the isoparaffin-rich liquid has a kinematic viscosity at 40° C. of 1 to 15 cSt (advantageously 2 to 10 cSt). In another embodiment, the isoparaffin-rich liquid has a kinematic viscosity at 25° C. of 1 to 30 cSt (advantageously 2 to 25 cSt, advantageously 3 to 20 cSt, advantageously 5 to 15 cSt) and a glass transition temperature (T_{g}) that cannot be determined by ASTM E 1356 or if it can be determined then it is less than 0° C. (advantageously less than −10° C., advantageously less than −20° C., advantageously less than −30° C.).

In another embodiment the isoparaffin-rich liquid has one or more of the following properties:
1. a pour point of −40° C. or less (advantageously −50° C. or less, advantageously −60° C. or less); and/or
2. a kinematic viscosity at 25° C. of from 1 to 30 cSt; and/or
3. a number average molecular weight (Mₙ) between 2,000 and 100 g/mol (advantageously between 1500 and 150 g/mol, advantageously between 1000 and 200 g/mol); and/or
4. a flash point (ASTM D 56 or D 93) of 50 to 200° C.; and/or
5. a specific gravity (15.6/15.6° C.) of less than 0.85 (advantageously less than 0.84, advantageously less than 0.83, advantageously from 0.65 to 0.85, advantageously from 0.70 to 0.84, advantageously from 0.75 to 0.83, advantageously from 0.800 to 0.840); and/or
6. a density of from 0.70 to 0.85 g/cm³.

Suitable isoparaffin-rich hydrocarbon liquids are described in, for example U.S. Pat. No. 3,818,105, U.S. Pat. No. 3,439,088 and U.S. Pat. No. 6,197,285, and are commercially available under the tradename ISOPAR™ (ExxonMobil Chemical). Other suitable isoparaffin-rich hydrocarbon liquids are commercial available under the trade names SHELLSOL™ (Royal Dutch/Shell), SOLTROL™ (Chevron Phillips) and SASOL™ (Sasol Limited).

ISOPAR ™ Series Isoparaffins

| | KV @ | pour point | specific | flash point |
|---|---|---|---|---|
| Grade | 25° C. (cSt) | (° C.) | gravity | (° C.) |
| H | 1.8 | −63 | 0.76 | 53 |
| K | 1.9 | −60 | 0.76 | 55 |
| L | 2.0 | −57 | 0.77 | 62 |
| M | 3.8 | −57 | 0.79 | 92 |
| V | 14.8 | −63 | 0.82 | 130 |

In another embodiment, the modifying fluid is a mixture of branched and normal paraffins having 6 to 50 (advantageously 8 to 40, advantageously 10 to 30) carbon atoms in the molecule, and has a ratio of branch paraffin to n-paraffin of 1:1 to 100:1 (advantageously 1:1 to 10:1). The distribution of branches in the isoparaffins of the mixture is such that at least 50% (advantageously at least 70%) are methyl branches, with less than 50% (advantageously less than 30%) of branches with carbon number greater than 1 (for example, ethyl, propyl, butyl or the like). These branch paraffin/n-paraffin blends are described in, for example, U.S. Pat. No. 5,906,727.

d. Lubricant Basestocks Derived from Gas-to-Liquids Processes:

In another embodiment, the modifying fluid is a high purity hydrocarbon fluid of lubricating viscosity comprising a mixture of C₂₀ to C₁₂₀ paraffins, 50 wt % or more being isoparaffinic hydrocarbons and less than 50 wt % being hydrocarbons that contain naphthenic and/or aromatic structures. Advantageously, the mixture of paraffins comprises a wax isomerate lubricant basestock or oil, which includes:
1. hydroisomerized natural and refined waxes, such as slack waxes, deoiled waxes, normal alpha-olefin waxes, microcrystalline waxes, and waxy stocks derived from gas oils, fuels hydrocracker bottoms, hydrocarbon raffinates, hydrocracked hydrocarbons, lubricating oils, mineral oils, polyalphaolefins, or other linear or branched hydrocarbon compounds with carbon number of about 20 or more; and
2. hydroisomerized synthetic waxes, such as Fischer-Tropsch waxes (i.e., the high boiling point residues of Fischer-Tropsch synthesis, including waxy hydrocarbons); or mixtures thereof. Most advantageous are lubricant basestocks or oils derived from hydrocarbons synthesized in a Fischer-Tropsch process as part of an overall Gas-to-Liquids (GTL) process.

In one embodiment, the mixture of paraffins has two or more of the following properties:
1. a naphthenic content of less than 40 wt % (advantageously less than 30 wt %, advantageously less than 20 wt %, advantageously less than 15 wt %, advantageously less than 10 wt %, advantageously less than 5 wt %, advantageously less than 2 wt %, advantageously less than 1 wt %) based on the total weight of the hydrocarbon mixture; and/or
2. a normal paraffins content of less than 5 wt % (advantageously less than 4 wt %, advantageously less than 3 wt %, advantageously less than 1 wt %) based on the total weight of the hydrocarbon mixture; and/or
3. an aromatic content of 1 wt % or less (advantageously 0.5 wt % or less); and/or
4. a saturates level of 90 wt % or higher (advantageously 95 wt % or higher, advantageously 98 wt % or higher, advantageously 99 wt % or higher); and/or
5. the percentage of carbons in chain-type paraffinic structures (C_{P}) of 80% or more (advantageously 90% or more, advantageously 95% or more, advantageously 98% or more); and/or
6. a branched paraffin:normal paraffin ratio greater than about 10:1 (advantageously greater than 20:1, advantageously greater than 50:1, advantageously greater than 100:1, advantageously greater than 500:1, advantageously greater than 1000:1); and/or
7. sidechains with 4 or more carbons making up less than 10% of all sidechains (advantageously less than 5%, advantageously less than 1%); and/or
8. sidechains with 1 or 2 carbons making up at least 50% of all sidechains (advantageously at least 60%, advantageously at least 70%, advantageously at least 80%, advantageously at least 90%, advantageously at least 95%, advantageously at least 98%); and/or
9. a sulfur content of 300 ppm or less (advantageously 100 ppm or less, advantageously 50 ppm or less, advantageously 10 ppm or less) where ppm is on a weight basis; and/or
10. a nitrogen content of 300 ppm or less (advantageously 100 ppm or less, advantageously 50 ppm or less, advantageously 10 ppm or less) where ppm is on a weight basis; and/or
11. a number-average molecular weight of 300 to 1800 g/mol (advantageously 400 to 1500 g/mol, advantageously 500 to 1200 g/mol, advantageously 600 to 900 g/mol); and/or
12. a kinematic viscosity at 40° C. of 10 cSt or more (advantageously 25 cSt or more, advantageously between about 50 and 400 cSt); and/or
13. a kinematic viscosity at 100° C. ranging from 2 to 50 cSt (advantageously 3 to 30 cSt, advantageously 5 to 25 cSt, advantageously 6 to 20 cSt, advantageously 8 to 16 cSt); and/or
14. a viscosity index (VI) of 80 or greater (advantageously 100 or greater, advantageously 120 or greater, advantageously 130 or greater, advantageously 140 or greater, advantageously 150 or greater, advantageously 160 or greater, advantageously 180 or greater); and/or
15. a pour point of −5° C. or lower (advantageously −10° C. or lower, advantageously −15° C. or lower, advantageously −20° C. or lower, advantageously −25° C. or lower, advantageously −30° C. or lower); and/or
16. a flash point of 200° C. or more (advantageously 220° C. or more, advantageously 240° C. or more, advantageously 260° C. or more); and/or
17. a specific gravity (15.6° C./15.6° C.) of 0.86 or less (advantageously 0.85 or less, advantageously 0.84 or less); and/or
18. an aniline point of 120° C. or more; and/or
19. a bromine number of 1 or less.

In an advantageous form, the mixture of paraffins comprises a GTL basestock or oil. GTL basestocks and oils are fluids of lubricating viscosity that are generally derived from waxy synthesized hydrocarbons, that are themselves derived via one or more synthesis, combination, transformation, and/or rearrangement processes from gaseous carbon-containing compounds and hydrogen-containing compounds as feedstocks, such as: hydrogen, carbon dioxide, carbon monoxide, water, methane, ethane, ethylene, acetylene, propane, propylene, propyne, butane, butylenes, and butynes. Advantageously, the feedstock is “syngas” (synthesis gas, essentially CO and H₂) derived from a suitable source, such as natural gas and/or coal. GTL basestocks and oils include wax isomerates, comprising, for example, hydroisomerized synthesized waxes, hydroisomerized Fischer-Tropsch (F-T) waxes (including waxy hydrocarbons and possible analogous oxygenates), or mixtures thereof. GTL basestocks and oils may further comprise other hydroisomerized basestocks and base oils. Particularly advantageous GTL basestocks or oils are those comprising mostly hydroisomerized F-T waxes and/or other liquid hydrocarbons obtained by a F-T synthesis process.

The synthesis of hydrocarbons, including waxy hydrocarbons, by F-T may involve any suitable process known in the art, including those involving a slurry, a fixed-bed, or a fluidized-bed of catalyst particles in a hydrocarbon liquid. The catalyst may be an amorphous catalyst, for example based on a Group VIII metal such as Fe, Ni, Co, Ru, and Re on a suitable inorganic support material, or a crystalline catalyst, for example a zeolitic catalyst. The process of making a lubricant basestock or oil from a waxy stock is characterized as a hydrodewaxing process. A hydrotreating step, while typically not required for F-T waxes, can be performed prior to hydrodewaxing if desired. Some F-T waxes may benefit from removal of oxygenates while others may benefit from oxygenates treatment prior to hydrodewaxing. The hydrodewaxing process is typically conducted over a catalyst or combination of catalysts at high temperatures and pressures in the presence of hydrogen. The catalyst may be an amorphous catalyst, for example based on Co, Mo, W, etc. on a suitable oxide support material, or a crystalline catalyst, for example a zeolitic catalyst such as ZSM-23 and ZSM-48 and others disclosed in U.S. Pat. No. 4,906,350, often used in conjunction with a Group VIII metal such as Pd or Pt. This process may be followed by a solvent and/or catalytic dewaxing step to lower the pour point of the hydroisomerate. Solvent dewaxing involves the physical fractionation of waxy components from the hydroisomerate. Catalytic dewaxing converts a portion of the hydroisomerate to lower boiling hydrocarbons; it often involves a shape-selective molecular sieve, such as a zeolite or silicoaluminophosphate material, in combination with a catalytic metal component, such as Pt, in a fixed-bed, fluidized-bed, or slurry type process at high temperatures and pressures in the presence of hydrogen.

Useful catalysts, processes, and compositions for GTL basestocks and oils, Fischer-Tropsch hydrocarbon derived basestocks and oils, and wax isomerate hydroisomerized basestocks and oils are described in, for example, U.S. Pat. Nos. 2,817,693; 4,542,122; 5,545,674; 4,568,663; 4,621,072; 4,663,305; 4,897,178; 4,900,407; 4,921,594; 4,923,588; 4,937,399; 4,975,177; 5,059,299; 5,158,671; 5,182,248; 5,200,382; 5,290,426; 5,516,740; 5,580,442; 5,885,438; 5,935,416; 5,935,417; 5,965,475; 5,976,351; 5,977,425; 6,025,305; 6,080,301; 6,090,989; 6,096,940; 6,103,099; 6,165,949; 6,190,532; 6,332,974; 6,375,830; 6,383,366; 6,475,960; 6,620,312; and 6,676,827; European Patents EP 324528, EP 532116, EP 532118, EP 537815, EP 583836, EP 666894, EP 668342, EP 776959; WPO patent applications WO 97/31693, WO 99/20720, WO 99/45085, WO 02/64710, WO 02/64711, WO 02/70627, WO 02/70629, WO03/33320; and British Patents 1350257; 1390359; 1429494; and 1440230. Particularly favorable processes are described in European Patent Applications EP 464546 and EP 464547. Processes using Fischer-Tropsch wax feeds are described in U.S. Pat. Nos. 4,594,172; 4,943,672; 6,046,940; 6,103,099; 6,332,974; 6,375,830; and 6,475,960.

As used herein, the following terms have the indicated meanings: “hydroisomerized” describes a catalytic process in which normal paraffins and/or slightly branched isoparaffins are converted by rearrangement into more branched isoparaffins (also known as “isodewaxing”); “wax” is a hydrocarbonaceous material existing as a solid at or near room temperature, with a melting point of 0° C. or above, and consisting predominantly of paraffinic molecules, most of which are normal paraffins; “slack wax” is the wax recovered from petroleum oils such as by solvent dewaxing, and may be further hydrotreated to remove heteroatoms.

e. High VI Paraffinic Mineral Oils:

In another form, the modifying fluid is a Group III Mineral Oil, with a saturates levels of 90% or more (advantageously 92% or more, advantageously 94% or more, advantageously 95% or more, advantageously 98% or more); a sulfur content of less than 0.03% (advantageously between 0.001 and 0.01%); and a VI of 120 or more (advantageously 130 or more, advantageously 140 or more). Advantageously the Group III Mineral Oil has a kinematic viscosity at 100° C. of 3 to 50, advantageously 4 to 40 cSt, advantageously 6 to 30 cSt, advantageously 8 to 20; and/or a number average molecular weight of 300 to 5,000 g/mol, advantageously 400 to 2,000 g/mol, advantageously 500 to 1,000 g/mol. Advantageously the Group III Mineral Oil has a pour point of −10° C. or less, a flash point of 200° C. or more, and a specific gravity (15.6° C./15.6° C.) of 0.86 or less.

Commercially available Group III Mineral Oils

| | KV @ | | Pour | | Flash |
|---|---|---|---|---|---|
| | 100° C., | | Point, | Specific | Point, |
| | cSt | VI | ° C., | gravity | ° C. |
| UCBO 4R¹ | 4.1 | 127 | −18 | 0.826 | 216 |
| UCBO 7R¹ | 7.0 | 135 | −18 | 0.839 | 250 |
| Nexbase 3043² | 4.3 | 124 | −18 | 0.831 | 224 |
| Nexbase 3050² | 5.1 | 126 | −15 | 0.835 | 240 |
| Nexbase 3060² | 6.0 | 128 | −15 | 0.838 | 240 |
| Nexbase 3080² | 8.0 | 128 | −15 | 0.843 | 260 |
| Yubase YU-4³ | 4.2 | 122 | −15 | 0.843 | 230 |
| Yubase YU-6³ | 6.5 | 131 | −15 | 0.842 | 240 |
| Yubase YU-8³ | 7.6 | 128 | −12 | 0.850 | 260 |
| Ultra-S 4⁴ | 4.3 | 123 | −20 | 0.836 | 220 |
| Ultra-S 6⁴ | 5.6 | 128 | −20 | 0.839 | 234 |
| Ultra-S 8⁴ | 7.2 | 127 | −15 | 0.847 | 256 |
| VHVI 4⁵ | 4.6 | 128 | −21 | 0.826 | |
| VHVI 8⁵ | 8.0 | 127 | −12 | 0.850 | 248 |
| Visom 4⁶ | 4.0 | | | | 210 |
| Visom 6⁶ | 6.6 | 148 | −18 | 0.836 | 250 |

¹Available from ChevronTexaco (USA).
²Available from Neste Oil (Finland).
³Available from SK Corp (South Korea).
⁴Available from ConocoPhillips (USA)/S-Oil (South Korea).
⁵Available from PetroCanada (Canada).
⁶Available from ExxonMobil (USA).

In another form, the modifying fluid is an Exceptional Paraffinic Process Oil, which is a mineral oil with
A) a viscosity index of 90 to 119, and
B) kinematic viscosity at 40° C. of 80 cSt or more (advantageously 90 cSt or more, advantageously 100 cSt or more, advantageously 120 cSt or more, advantageously 150 cSt or more, advantageously 200 cSt or more, advantageously 250 cSt or more, advantageously 300 cSt or more), and
C) a pour point of −15° C. or less (advantageously −18° C. or less, advantageously −20° C. or less, advantageously −25° C. or less, advantageously −30° C. or less, advantageously −35° C. or less).

Commercially available Exceptional Paraffinic Process Oils

| | KV @ | KV @ | | Pour | | Flash |
|---|---|---|---|---|---|---|
| | 40° C. | 100° C. | | Point | Specific | Point |
| | cSt | cSt | VI | ° C. | gravity | ° C. |
| Paralux 6001R¹ | 118 | 12 | 102 | −21 | 0.875 | 274 |
| Diana PW90² | 90 | 11 | 105 | −22 | 0.872 | 262 |
| Diana PW380² | 376 | 26 | 106 | −19 | 0.877 | 293 |

¹Available from Chevron (USA).
²Available from Idemitsu (Japan).

In certain forms, paraffinic mineral oils with a kinematic viscosity at 40° C. of less than 80 cSt and a pour point of greater than −15° C. are substantially absent from the compositions of the present invention.

f. Esters:

Other fluids can be used in this invention are disclosed herein. Exemplary fluids are described in the book SYNTHETICS, MINERAL OILS AND BIO-BASED LUBRICANTS, CHEMISTRY AND TECHNOLOGY, ed. by L. R. Rudnick, published in 2006 by CRC Press, Taylor & Francis Group, Boca Raton, Fla.

Esters or polyester fluids, described in Chapter 3 and 5 of this book and made from aliphatic or aromatic mono-basic, di-basic, multi-basic acids with different alcohols, such as linear, branched, polyols, aromatic alcohols, may be used in the functional polymer matrix. The ester fluids of 2 to 500 cS may be suitable. Non-limiting exemplary ester fluids include maleates, adipates, azelates, sebacates, laureates, oleates, benzoates, phthalates, terephthalates, isophthalates, trimellitates, pyromellitate, dimerates, and esters of C₁ to C₂₂-aliphatic mono-acids. Alternatively, mixtures of the acids may be used to improve product properties, such as low temperature performance, material cost or volatility. Non-limiting exemplary alcohols include linear or branched C₁-C₃₆ mono-alcohols, 2-ethylhexanol, iso-heptyl alcohol, iso-octyl alcohol, iso-nonyl alcohol, iso-decyl alcohol, iso-tridecyl alcohol, pentaerythritol (PE), dipentaerythritol (DPE), trimethylolpropane (TMP), and neopentylglycol (NPG).

Mixed alcohols may also be used with acids to improve ester properties. For example, iso-tridecanol is available in a mixture of branched carbon chain lengths in the range of C₁₁ to C₁₄, rich in C₁₃ alkyl chain, Iso-decanol is available in a mixture of branched carbon chain of C₉ to C₁₁, rich in C₁₀ alkyl chains. Different alcohols may be mixed together to react with acids to produce esters of the most desirable properties. A small amount of hydroxyl group may also be present in the final ester fluid to improve fluid properties. Alcohols of 3 to 50 cS fluids made from these acids and alcohols are available from ExxonMobil Chemical Company, Houston, USA. The esters described above may be used as the fluids to carry active agents in the polymer matrix, similar to the PAO components described earlier. Often, it is more beneficial to use a mixture of ester and PAO as the fluid components. The addition of ester fluid to PAO may impart a unique property not available from a pure PAO fluid. For example, the ester may improve the compatibility of the PAO with the base polymer. Alternatively, the ester may improve the compatibility of the fluid components with the active agents, thus facilitating the process of blending the active substrate into the polymer matrix. Alternatively, the ester can improve the dispersion of the active agents in the polymer matrix to improve the effectiveness of the active agents. Alternatively, the ester can improve the controlled release of the fluids and the active agents from the polymer matrix to the surface. All these are factors to ensure effective usage of the active agents, which is typically most expensive in the finished product formulation. The amount of ester that may be added to the PAO fluids for incorporation in the functional polymer compositions disclosed herein may range from 0.1 wt % to 100%, or from 1 wt % to 50 wt %, or from 1 wt % to 25 wt %, or from 1 wt % to 20 wt %, or from 2 wt % to 10 wt %. For simplicity of operation, the one or more esters and one or more PAO fluids may be pre-mixed before addition of the active agents or alternatively all fluids and active agents may be added together to given a liquid component, which can then be added to the base polymer during processing. Alternatively, all fluids, active agents and base polymers can be added to the processing apparatus at the same time. Heat may be applied to facilitate the solubility or mixing of all the ingredients.

g. Other fluids:

Other fluids similar to the ester fluids described above may be used in functional polymer compositions disclosed herein. For example, polyalkylene glycol (PAG) made from aliphatic and/or aromatic epoxides, as described in Chapter 8 of “Synthetics, Mineral Oils and Bio-Based Lubricants, Chemistry and Technology,” may be used in functional polymer compositions disclosed herein. Alternatively, aliphatic and/or aromatic silicone fluids, as described in Chapter 12 of SYNTHETICS, MINERAL OILS AND BIO-BASED LUBRICANTS, CHEMISTRY AND TECHNOLOGY, may also be in functional polymer compositions disclosed herein. Other non-limiting exemplary fluids include alkylated aromatics, polyinternal olefins, polychlorotrifluoroethylene, dialkyl bonates, poly-1-butene liquids, lube fractions produced in gas to liquid process technology, and chemically modified mineral oils. These fluids are of generally 2 to 3000 cS viscosity and may be used individually or in combination with one another (referred to herein as fluid mixtures) in the polymer matrix. Non-limiting exemplary fluid mixtures include PAO with esters, PAO with silicone fluids, PAG fluid with PAO fluid, PAG with esters, PAG with alkylated aromatics, esters with polybutenes, PAO/ester/PAG mixtures, PAO/ester/silicone fluid mixtures, and ester/PAG/silicone fluid mixtures.

These fluids and fluid mixtures sometimes impart a special function. For example, PAO, PAG or silicone fluids result in a unique moisturizing effect. When they are added to the polymer matrix and are being slowly released to the surface, the surface components can act as a moisturizing agent to the user. Or when active agents are mixed with these fluids, the active agents can be released from the polymer matrix in a controlled manner with the migration of these fluids. The amount of these fluids incorporated into the polymer matrix may generally range from 0.1 wt % to 50 wt %, usually advantageously from 0.3 to 30 wt %, from 0.5 to 25 wt %, from 0.1 to 20 wt %, from 0.1 to 10 wt %, or from 0.1 to 5 wt %. The amount to be incorporated depends on the function it is to be serviced as will be exemplified from the specific embodiments which follow.

### Active Substrates:

One or more active substrate(s) may be included in the functional polymer compositions to impart special functionality. Exemplary active agents include, but are not limited to, moisturizing agents, anti-bacterial agents, anti-septic agents (also referred to as disinfecting agents), anti-viral agents, anti-mildew agents, anti-mold agents, anti-fungal agents, anti-microbial agents, moisture/odor absorbing agents (deodorant agents), fragrance agents, insect repellant agents, anti-static agents, vitamin agents and combinations thereof.

The amount of these active substrates incorporated into the polymer matrix may generally range from 0.1 wt % to 50 wt %, usually advantageously from 0.3 to 30 wt %, from 0.5 to 25 wt %, from 0.1 to 20 wt %, from 0.1 to 10 wt %, or from 0.1 to 5 wt %. The amount to be incorporated depends on the special functionality desired as will be exemplified from the specific embodiments which follow.

Moisturizing agents include, but are not limited to, emollients, silicones, petrolatum, esters, aloe, natural moisturizing agents, glycerol and combinations thereof. Anti-septic agents include, but are not limited to, ethanol, isopropanol, alcohols, zinc acetate, hexachlorophene, 4-hexylresorcinol and combinations thereof. Anti-microbial agents (also referred to as an anti-bacteria agent), include, but are not limited to, triclosan, Zn containing compounds, peroxide containing compounds and combinations thereof. Vitamin agents include vitamins A, B complexes, C, E, K and combinations thereof.

### Solid Carrier Agents:

In addition, the functional polymer compositions may optionally include a mesoporous material containing active substrate for controlled release or slow release, a mesoporous material for absorbing undesirable components, and combinations thereof. Non-limiting exemplary mesoporous materials include zeolites, natural or synthetic clay, alumina, silica, and silica-aluminate. Specific examples of useful solid carrier agents include MCM41, MCM49, MCM56, MCM36, SBA-15, and FSM-16. U.S. Pat. Nos. 5,145,816, 5,925,330, 5,098,684 and 5,102,643 describe in detail mesoporous materials and in particular MCM41, and are herein incorporated by reference in their entirety. Other porous materials, such as natural or synthetic clay, alumina, silica, silica-aluminate, etc. are also suitable for this application. Generally, porous material with high pore volume, large pore sizes to absorb the fluids and the active substrates are advantageous for this use. Zeolites of large enough pores and high pore volumes are also suitable. Non-limiting examples of these zeolites includes MCM22, MCM36, MCM49, MCM56, etc. Descriptions of these porous materials may be found in U.S. Pat. Nos. 4,954,325, 5,292,698, 5,236,575, and 5,362,697, herein incorporated by reference in their entirety.

One non-limiting exemplary method to incorporate the solid mesoporous or microporous materials is to impregnate the fluid with or without active substrates into the solid material by impregnation methods. In this method, all the fluids with or without active substrates of proper amount are dissolved in a common solvent. This solution may then be mixed with the solid mesoporous or microporous material in a proper proportion and stirred for a sufficient time to allow the fluid and active substrate(s) to diffuse into the porous material. The liquid can then be removed by filtration, by evaporation under vacuum or by a purge gas. The final dried porous material can then be incorporated into the base polymer for controlled release. Alternatively, the solid mesoporous or microporous material and modifying fluid(s) with or without active substrate(s) can be stirred together to yield the porous active material, which can then be incorporated into the base polymer for controlled release.

In one form of the functional polymer compositions disclosed herein, a polyolefin based polymer includes a liquid PAO fluid and optional ester fluid, such that some of the fluids are present at the surface of a part formed from the polymer composition and can be transferred to the surface of the skin for acting as moisturizer. In another form of the functional polymer compositions disclosed herein, the polyolefin based polymer includes one or more fluids and also contains an anti-microbial agent, such as triclosan. In another embodiment, a fragrance is dissolved in one or more fluids, which is then incorporated into the functional polymer composition. The presence of fluid facilitates the transport of the anti-microbial agent or the fragrance to the surface of a part or article formed from the functional polymer composition to provide enhanced or prolonged activity than without the fluid. Alternatively, the fluid component continuously diffuses to the surface of the part or article formed from the functional polymer composition within the life time of the part or article, thus prolonging the active function.

Controlled Release of Fluids and/or Active Substrates:

As previously described, the one or more fluids and/or one or more active substrates included within the functional polymer matrix disclosed herein migrate through the polymer matrix at a controlled rate. The controlled migration or release rate provides for a time-release aspect of the functional attribute(s) provided by the one or more fluids with or without one or more active substrates. The controlled migration rate may vary from little to no release to the surface of the article formed from the functional polymer compositions to a high degree of release to the surface of the article formed from the functional polymer compositions. The release rate may be qualified as low, medium and high for the one or more fluids and/or one or more active substrates included within the functional polymer compositions disclosed herein.

The controlled release of the fluids and the active agents in a polymer matrix is a very complex process. Many factors affect this release phenomenon. In order to release these components from the polymer matrix, the fluids and the active agent must migrate to the surface from interior or the matrix. This migration rate depends on many factors. Diffusion rates of the fluids and the active components in the polymer matrix are part of the key controlling factors. These diffusion rates can be partially represented by the conventional diffusion laws, such as the Frick's first and second law of diffusion. Many accompanying physical processes which occur simultaneously with this diffusion, such as change of state, crystallization, formation of elastic stresses, loss of matrix material, etc. also affect the diffusion rates.

This diffusion rates is strongly influenced by the choice of components of the polymer matrix. Fluid molecules or active agent molecules with more compact shape or more spherical shape diffuse slower than their analogs with more elongated shape. For the same class of fluids or active agents, components with high volatility have higher diffusion rate. Components of higher molecular weights have lower diffusion rates. The rate of migration of the fluids and active agents also depends on their initial weight fraction in the polymer matrix. Higher loading usually promotes higher migration and higher release rates. Higher temperature of the polymer article also promotes higher migration and higher release rates.

The compatibility of base polymer with the fluid and active agents is another factor controlling the migration and release rates. If the fluids/active substrates have good compatibility and good wetting characteristics for the fluids/active substrates/polymer matrix surfaces, the release rate maybe higher. The release rate can be described by the following equation: W═P/(2MkT)^{1/2}, where P=partial pressure of fluids at temperature T, M=average molecular weight of fluids, k=rate constant of transfer of low molecular weight fluids from polymer matrix, and T=temperature.

The nature of the base polymer affects the release rate. For the same class of system, the base polymer with less compatibility with PAO and/or esters and/or active substrates may result in higher migration or release rates of the active substrates and fluids from the polymer matrix. Higher compatibility of base polymer with fluids or active substrates usually means that they have better interaction, and thus better solubility in each other. The thickness of the polymer matrix also affects the migration rate. The fluids and active substrates may take a longer time to migrate to the surface of the polymer matrix than a thinner polymer matrix.

The method used to fabricate the polymer composition can have a significant impact on the rate of controlled release. For example, in compression molding, after the polymer matrix was released from the mold, the rate of cooling effects the rate of release and the total releasable amount. Faster cooling often promotes faster release. Slower cooling promotes slower release, coupled with a lower total releasable amount of active ingredients.

For the same class of base polymers, the molecular weight, molecular weight distribution, fraction of amorphous and crystalline regions, crystallite size and shape, Tg and Tm, all have effects on the amount of fluids/active substrates that can be incorporated into the polymer matrix and the release rates. Polymers with a lower Tg, lower molecular weight and higher proportion of amorphous regions typically have a more movable polymer backbone. More flexible polymer backbones usually promote faster migration or release of the fluids or active substrates in the polymer matrix.

After the fluids and the active substrates are incorporated into the polymer matrix and fabricated into useful articles, the surface of the polymer matrix or the article can be further treated to decrease the amount of fluids or active substrate migration to the surface. This can be achieved by partial cross-linking via a chemical or electronic beam or other radiation treatment, by partial melting via high temperature treatment with hot air or hot surface, etc., by laminating with other polymers containing little or no fluids and/or active substrates. This may prevent unintended release of fluids and active substrates. When release is needed, this blocking surface can be removed by physical or chemical means to initiate the control release.

After the fluids and the active substrates are incorporated into the polymer matrix and fabricated into useful articles, the fluids and the active substrates are dispersed in the matrix. In the desirable application, these fluids and substrates should stay in place without any action until their functions are needed. When their functions are called for, a trigger action can be applied to the article to initiate this release process. Non-limiting exemplary release triggers may be heat (such as body heat, exposure to sunlight, deliberate heating by external source, exposure to heat outside), or pressure (such as pressure exerted by body actions, by gravity, by rubbing), heat loss (such as exposure to external cold temperature), chemical agents (such as exposure to ambient air or moisture, or high amount of CO₂, etc.). Other release-activating mechanisms can also be used.

In one form, the one or more fluids may also be used to assist with controlled migration of the one or more active substrates within the functional polymer composition. In particular, the one or more fluids may act as carriers for the transport of the one or more active substrates in the functional polymer composition to allow for controlled time release migration of the active substrates from the bulk of the polymer composition to the surface of the functional polymer composition. This time released aspect permits the functionality to be sustained within the functional polymer composition for extended periods of time. In another form, the one or more fluids may be used to both impart special functionality to the functional polymer composition and also to provide for controlled migration or release of the fluid from the bulk of the polymer matrix to the surface of the polymer matrix.

Below are described numerous non-limiting exemplary embodiments of the functional polymer compositions disclosed herein. The embodiments described may be made as either a polymer composition for subsequent part or article formation via melt processing or may be made directly into the part or article. Exemplary parts or articles, include, but are not limited to, extruded products, molded products, films, fibers, and fabrics (woven and non-woven), which will be described in greater detail below.

Embodiments of Functional Polymer Compositions with Moisturizing Effect:

In exemplary embodiment 1 of a functional polymer composition with a moisturizing effect, a synthetic polymeric resin component may be mixed with 1-50 wt %, or 2-40 wt %, or 5-30 wt % or 10-20 wt % of a fluid, for example 4 to 1000 cS PAO fluid, in a batch mixer or extruder. The particular concentration range utilized is dependent on the type of fluid used. The mixture may then be melt processed to produce an extruded product, a molded product, film, a fiber, a non-woven fabric or other article in such a manner that part of the PAO is maintained at or close to the surface of the article. When formed into a fabric, it may be fabricated alone or laminated with other fabrics into gloves, face masks, socks, underpants, undershirts, etc. These articles will have a continuous moisturizing effect during its normal usage. This resin component may be polyethylene, polypropylene, polyethylene propylene elastomers or other previously disclosed polyolefin component. Examples of these polymers may be found in the detailed description of International Patent Publication No. WO2004014998, pages 29 to page 61, herein incorporated by reference.

Exemplary embodiment 2 of a functional polymer composition with a moisturizing effect is similar to exemplary embodiment 1, except that during the processing, the fluid may contain a mixture of 4-1000 cS PAO with 1 to 90 wt % or 2 to 50 wt % or 5 to 30 wt % or 10 to 20 wt % of petrolatum. This fluid mixture may be used to produce the fabric or other article.

Exemplary embodiment 3 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % aloe.

Exemplary embodiment 4 of a functional polymer composition with a moisturizing effect is similar to exemplary embodiment 2, except the fluid may contain a mixture of 4-1000 cS PAO and 1-50% or 2 to 40 wt % or 5 to 30 wt % or 10 to 20 wt % PureSyn™ ester.

Exemplary embodiment 5 of a functional polymer composition with a moisturizing effect is similar to exemplary embodiment 2, except the fluid may contain a mixture of 4-1000 cS PAO and 1-50% or 2 to 40 wt % or 5 to 30 wt % or 10 to 20 wt % dimethylcone and/or cyclomethicone and/or other silicone fluids.

Exemplary embodiment 6 of a functional polymer composition with a moisturizing effect is similar to exemplary embodiment 2, except the fluid may contain a mixture of 4-1000 cS PAO and 1-50% or 2 to 40 wt % or 5 to 30 wt % or 10 to 20 wt % fatty alcohols (cetyl alcohol, stearyl alcohol or ceteary alcohol).

Exemplary embodiment 7 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % other hydrocarbon moisturizers (mineral oil, squalane, squalene, microcrystalline wax).

Exemplary embodiment 8 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % polyols (propylene glycol, glycerine, sorbitol, butylenes glycol, ethylene oxide-propylene oxide copolymer, polyalkylene glycol (PAG).

Exemplary embodiment 9 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % PureSyn™ ester.

Exemplary embodiment 10 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % esters (isopropyl myristate, palmitate, state, octyl palmitate, jojoba or mixture of nature esters).

Exemplary embodiment 11 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % natural oils (vegetable oils, seed oils).

Exemplary embodiment 12 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % other typical moisturizers.

Exemplary embodiment 13 of a functional polymer composition with a moisturizing effect is similar to exemplary embodiment 2, except the fluid may contain a mixture of 4-1000 cS PAO and 1-50 wt % or 2 to 40 wt % or 5 to 30 wt % or 10 to 20 wt % of at least two of any of the above moisturizing components.

Exemplary embodiment 14 of a functional polymer composition with a moisturizing effect is similar to exemplary embodiment 2, except a resin component may be mixed with 1-50 wt % or 2 to 40 wt % or 5 to 30 wt % or 10 to 20 wt % of a fluid, 4 to 1000 cS PIB fluid, in a mixer.

Exemplary embodiment 15 of a functional polymer composition with a moisturizing effect is similar to exemplary embodiment 2, except a resin component may be mixed with 1-50 wt % or 2 to 40 wt % or 5 to 30 wt % or 10 to 20 wt % of a 4 to 1000 cS polyinternal olefin fluid, in a mixer.

Exemplary embodiment 16 of a functional polymer composition with a moisturizing effect is similar to exemplary embodiment 2, except the resin may be first processed into a melt processed article. The PAO liquid or the mixture of liquid may then be impregnated or sprayed onto the surface of the article, such as a fabric. The liquid forms a thin film at the surface or partially penetrates into the article structure for the case of a fabric. This surface liquid film will provide a moisturizing effect during use to the fabric. The liquid that is penetrated into a fabric article may slowly migrate to the surface and provide a moisturizing effect continuously.

Embodiments of Functional Polymer Compositions with Anti-Bacteria Effect:

Exemplary embodiment 17 of a functional polymer composition with anti-bacteria effect is similar to exemplary embodiments 1 to 16, except the fluid contains a fluid or a mixture of fluids and one or more anti-bacteria agents. The concentration of the fluids or a mixture of fluids and one or more anti-bacteria agents may range from 0.01 to 20 Wt %, or 0.05 to 10 wt % or 0.1 to 5 wt % or 0.2 to 2 wt %. The particular concentration range utilized is dependent on the type of anti-bacteria agents used. Non-limiting examples of the anti-bacteria agents include triclosan, triclocarban, families of antibacterial agents derived from quinolones, nitrofurans, sulfonamides, families of stable anti-biotics as described in Kirk-Othmer Encyclopedia of Chemical Technology, 4^{th} edition, volume 2, p. 854-893. These active substrate compounds are mixed with fluids containing PAO and/or other fluid components. These active substrates agents are either soluble, or form an emulsion or highly dispersed suspension with the fluids at room temperature or a slightly higher temperature. Other promoting agents, e.g. EDTA, may be added to further enhance the function. After melt processing into an article, the part of the fluids and active substrates are at the surface of the article or embedded inside the article. They may function to prevent bacteria growth with slow release over time on the article, such as a fabric. Alternatively, when the components are transferred to the user skin or other contacting surface in the case of a fabric article, it continues to provide anti-bacteria effect. In one particularly advantageous form of this embodiment, triclosan or tetracycline in PAO and/or ester in resins or fabrics may be utilized.

In a similar exemplary embodiment the fluid used is a mixture containing 80% of a 10 cS PAO and 20% of a dibasic ester, di-tridecyladipate.

In yet another exemplary embodiment, the fluid used is a mixture containing 80% of a 10 cS PAO and 20% of a phthalate ester, di-tridecylphthalate.

Embodiments of Functional Polymer Compositions with Disinfecting Effect:

Exemplary embodiment 18 of a functional polymer composition with disinfecting agents is similar to exemplary embodiments 1 to 17, except the fluid contains a fluid or a mixture of fluids and one or more antiseptic agents (or disinfectants). The concentration of the fluids or a mixture of fluids and one or more disinfecting agents may range from 0.01 to 95 wt %, or 0.05 to 50 wt % or 0.1 to 20 wt % or 0.2 to 10 wt %. The particular concentration range utilized is dependent on the type of antiseptics used. Non-limiting exemplary antiseptic agents or disinfectants include ethanol, isopropyl alcohol, butanol, other alcohols, zinc compounds (zinc oxide, zinc halides, zinc acetate, zinc citrate, zinc gluconate, etc.), hexachlorophene, 4-hexylresorcinol or phenolic derivatives, hydroxybenzoic acids and esters or their derivatives, salicylic acid or its derivatives, per-acids or their derivatives, quaternary ammonium compounds, surfactant disinfectants, peroxide compounds etc. Families of other useful stable antiseptic or disinfectant agents are described in KIRK-OTHMER ENCYCLOPEDIA OF CHEMICAL TECHNOLOGY, 4^{th} edition, volume 8, p. 237-292. These active substrate compounds may be mixed with fluids containing PAO and/or other fluid components. These active substrates are either soluble, or form an emulsion or highly dispersed suspension with the fluids at room temperature or slightly higher temperature. Other promoting agents, e.g. EDTA, may be added to further enhance the function. After processing into fabric or other article, the part of the fluids and active substrates may be at the surface of the fabric/article or embedded inside the fabric/article. These fluids and active substrates function to prevent bacteria growth with slow release over time on the fabric/article. Alternatively, when the components are transferred to the user skin or other contacting surface, it continues to provide anti-bacteria effect.

Embodiments of Functional Polymer Compositions with Anti-Viral Effect:

Exemplary embodiment 19 of a functional polymer composition with anti-viral effect is similar to exemplary embodiments 1 to 18, except the fluid contains a fluid or a mixture of fluids and one or more antiviral agents. The concentration of the fluids or a mixture of fluids and one or more anti-viral agents may range from 0.01 to 20 wt %, or 0.05 to 10 wt % or 0.1 to 5 wt % or 0.2 to 2 wt %. The particular concentration range utilized is dependent on the type of anti-viral agents used. Non-limiting exemplary antiviral agents are amantadine, rimantadine and their derivatives. Families of other useful antiviral agents are described in KIRK-OTHMER ENCYCLOPEDIA OF CHEMICAL TECHNOLOGY, 4^{th} edition, volume 3, p. 567-607.

Embodiments of Functional Polymer Compositions with Anti-Mildew Effect:

Exemplary embodiment 20 of a functional polymer composition with anti-mildew effect is similar to exemplary embodiments 1 to 19, except the fluid contains a fluid or a mixture of fluids and one or more anti-mildew agents. The concentration of the fluids or a mixture of fluids and one or more anti-mildew agents may range from 0.01 to 20 wt % or 0.05 to 10 wt % or 0.1 to 5 wt % or 0.2 to 2 wt %. The particular concentration range utilized is dependent on the type of anti-mildew agents used. Non-limiting exemplary anti-mildew agents include p-dichlorobenzene, paraformaldehyde, wax or silicone resins that prevents moisture penetration, salicyanilide in IPA, oxalic acid, potassium or calcium sorbated, sorbic acid, boric acid or its derivatives, sodium or zinc pyrithione, amantadine, and combinations thereof.

Embodiments of Functional Polymer Compositions with Anti-Fungal Effect:

Exemplary embodiment 21 of a functional polymer composition with anti-fungal effect is similar to exemplary embodiments 1 to 20, except one or more anti-fungal agents are used The concentration of the anti-fungal agents may range from 0.01 to 20 wt % or 0.05 to 10 wt % or 0.1 to 5 wt % or 0.2 to 2 wt %. The particular concentration range utilized is dependent on the type of anti-fungal agent used. Non-limiting examples of anti-fungal agents include allylamine, azoles, polyenes, ciclopirox olamine, undecylenate, and combinations thereof. More discussion about anti-fungal components may be found in the book ANTIFUNGAL AGENTS, ADVANCES AND PROBLEMS by Kathrin Tintelnot, published by Springer Verlag.

Embodiments of Functional Polymer Compositions with Moisture/Odor Absorbing Effect:

Exemplary embodiment 22 of a functional polymer composition with moisture/odor absorbing effect is similar to exemplary embodiments 1 to 21, except one or more deodorant and/or moisture absorbing components are used. The concentration of the moisture/odor absorbing agents may range from 0.01 to 20 wt % or 0.05 to 10 wt % or 0.1 to 5 wt % or 0.2 to 2 wt %. The particular concentration range utilized is dependent on the type of moisture/odor absorbing agents used. Non-limiting examples of deodorant or moisture absorbing components include aluminum chlorohydrate, aluminum zirconium chloride hydroxide complex, activated aluminum chlorohydrate and aluminum zirconium chloride hydroxide complex, zinc phenolsulfonate, p-chloro-m-xylenol, and combinations thereof. Other examples may be found in KIRK-OTHMER ENCYCLOPEDIA OF CHEMICAL TECHNOLOGY, 4^{th} edition, volume 7, p. 601-602.

Embodiments of Functional Polymer Compositions with Fragrance Effect:

Exemplary embodiment 23 of a functional polymer composition with fragrance effect is similar to exemplary embodiments 1 to 22, except the functional agent is a fragrance component or a mixture of fragrance components. The concentration of the one or more fragrance agents may range from 0.01 to 20 wt % or 0.05 to 10 wt % or 0.1 to 5 wt % or 0.2 to 2 wt %. The particular concentration range utilized is dependent on the type of fragrance agent used. Non-limiting exemplary fragrance agents include citronellene, citronellol, citronellal, citral, linalool, menthol, (R)-(+)-β-citronellol, (S)-(+)-β-citronellol, geraniol, their ester derivatives, jasmone, terpenes and their derivatives, 2-phenylethanol and its derivatives, benzaldehyde and its derivative including cinnamaldehyde, and combinations thereof. The fabrics prepared in this manner may be fragrant for prolonged length of time because the fragrance is present both at the surface of the fabric or embedded inside the fabric for slow controlled release during the lifetime of the fabric. Examples of other fragrance or mixture of fragrant agents can be found in the book “THE CHEMISTRY OF FRAGRANCES, complied by D. H. Pybus and C. S. Sell, The Royal Society of Chemistry, 1999 Paperbacks.

Embodiments of Functional Polymer Compositions with Insect Repellant Effect:

Exemplary embodiment 24 of a functional polymer composition with insect repellant effect is similar to exemplary embodiments 1 to 23, except the functional agent is an insect repellant and/or promoter, which promotes the function of the insect repellant. The concentration of the one or more insect repellant agents may range from 0.01 to 20 wt % or 0.05 to 10 wt % or 0.1 to 5 wt % or 0.2 to 2 wt %. The particular concentration range utilized is dependent on the type of insect repellant agent used. Non-limiting exemplary insect repellants include N,N-diethyl-m-toluamide (DEET), dimethyl phthalate, 2-ethyl-1,3-hexadiol, indalone and combinations thereof. The fabrics or other articles prepared in this manner may have prolonged insect repellant property because the insect repellant is present both at the surface of the fabric/article for immediate release and/or embedded inside the fabric/article for slow controlled release during the lifetime of the fabric/article. Examples of other insect repellant or mixture of insect repellant and promoter agents can be found in KIRK-OTHMER ENCYCLOPEDIA OF CHEMICAL TECHNOLOGY, 4^{th} edition, volume 14, p. 589-592.

Embodiments of Functional Polymer Compositions with Antistatic Effect:

Exemplary embodiment 25 of a functional polymer composition with antistatic effect is similar to exemplary embodiments 1 to 24, except the functional agent is an antistatic agent and/or promoter, which promotes the antistatic function. The concentration of the one or more antistatic agents may range from 0.01 to 20 wt % or 0.05 to 10 wt % or 0.1 to 5 wt % or 0.2 to 2 wt %. The particular concentration range utilized is dependent on the type of antistatic agent used. Non-limiting examples of antistatic agents include nitrogen compounds such as long-chain amines, amides and quaternary ammonium salts; esters of fatty acids and their derivatives, sulfonic acids, alkyl aryl sulfonates; polyoxyethylene derivatives; polyalkylene glycols and their derivatives, polyhydric alcohols and their derivatives, phosphoric acid derivatives, molten salts, water, and combinations thereof. The fabrics or other articles prepared in this manner will have prolonged antistatic properties because the antistatic agent is present both at the surface of the fabric/article for immediate activity and/or embedded inside the fabric/article for slow controlled release during the lifetime of the fabric/article. Examples of other antistatic agents can be found in KIRK-OTHMER ENCYCLOPEDIA OF CHEMICAL TECHNOLOGY, 4^{th} edition, volume 3, p. 540-575.

Embodiments of Functional Polymer Compositions with Combined Effects:

Exemplary embodiments 1 to 25 may be combined to produce further embodiments of the functional polymer compositions having more than one effect in the functional polymer. For example, combinations of two or more of moisturizing, anti-bacterial (anti-biotic or anti-septic), disinfecting, anti-viral, anti-mildew, anti-fungal, moisture/odor absorbing, fragrance, insect repellant, and antistatic effects may be produced through combining the proper fluids and active agents. In one exemplary embodiment of a functional polymer with a combined effect, the functional polymer composition may have moisturizing and fragrance properties through the incorporation of one or more moisturizing agents and one or more fragrance agents. In another exemplary embodiment of functional polymer compositions with a combined effect, the fabric or other article formed from such compositions may have an insect repellant property and an anti-septic and/or anti-microbial property. In yet another exemplary embodiment of functional polymer compositions with a combined effect, the fabric or other articles from such compositions may have an anti-fungal property and an insect repellant property.

### Methods of Making Functional Polymer Compositions:

The functional polymer compositions disclosed herein are not limited by any particular method for forming the compositions. For example, the functional polymer compositions may be made by a variety of fabrication techniques including, but not limited to, extrusion compounding, and batch mixing. In particular, single screw and twin screw extrusion compounding may be used to combine to the one or more base polymer components, one or more active substrates, and/or one or more fluids that impart improved functionality to the polymer compositions and products produced there from. Alternatively, a batch type mixer or blender (Henschel, ribbon or other type) may be used to combine to the one or more base polymer components, one or more active substrates, and/or one or more fluids that impart improved functionality to the polymer compositions and products produced there from. In one advantageous process, the functional polymer compositions are formed via a twin screw extrusion compounding process. In another advantageous process, the functional polymer compositions are formed via a single screw extrusion compounding process. In another form, solution processing may be used to combine the one or more base polymer components, one or more active substrates, and/or one or more fluids. In solution processing the one or more base polymer components are dissolved in a suitable solvent followed by the addition of the active substrates and/or fluids.

In another form, the functional polymer compositions disclosed herein may be made by mixing all the components (one or more base polymers, one or more fluids, and one or more active substrates) during the melt fabrication process (extruding, molding, film, fiber, or fabric forming, melt blown and spunbonding, etc.) to form the finished product (extruded part, molded part, film, fiber, non-woven fabric). In another form, the one or more fluids may be pre-mixed with the one or more active substrates, and then mixed with the one or more polymer components prior to or during the melt fabrication process. In yet another form, the one or more fluids and/or active substrates may be made into master batch with a suitable carrier polymer resin and then added to the base polymer resin prior to or during melt fabrication process. In still yet another form, the one or more fluids may be mixed with one or more active substrates and then sprayed on or impregnated onto the extruded part, molded part, film, fiber, or fabric after the fabrication process to avoid putting the fluids and/or active substrates through a heat history required to form the base polymer resin into suitable form.

### Applications for Functional Polymer Compositions:

The functional polymer compositions disclosed herein may be used in any known thermoplastic or elastomer application involving film, fiber, and/or fabric formation and articles made from films, fibers, and/or fabrics. Non-limiting examples include uses in molded parts, extruded parts, films, tapes, sheets, tubing, hose, sheeting, wire and cable coating, adhesives, shoe soles, bumpers, gaskets, bellows, films, fibers, elastic fibers, nonwovens, spunbonds, sealants, surgical gowns and medical devices.

In particular, the functional polymer compositions disclosed herein are advantageous when formed into non-woven fabrics by methods described in Exemplary Embodiment 1 to 3 above, or by methods described in THE NON-WOVEN FABRICS HANDBOOK, by Ian Butler, published by INDA of 1200 Crescent Green, Suite 100, Cary, N.C. 27511, PO Box 1288 Cary N.C. 28512-1288, phone 919-233-1210, fax 919-233-1282, 1999. The non-woven fabrication method to produce functional non-woven fabrics is particularly advantageous.

### Adhesives

The functional polymer compositions disclosed herein may be used as adhesives, either alone or combined with tackifiers. The tackifier is typically present at about 1 wt % to about 50 wt %, based upon the weight of the blend, or wt % to 40 wt %, or 20 wt % to 40 wt %. Other additives, as described above, may also be added.

The adhesives can be used in any adhesive application, including but not limited to, disposables, packaging, laminates, pressure sensitive adhesives, tapes labels, wood binding, paper binding, non-wovens, road marking, reflective coatings, and the like. In some forms, the adhesives of this invention can be used for disposable diaper and napkin chassis construction, elastic attachment in disposable goods converting, packaging, labeling, bookbinding, woodworking, other assembly applications. Applications include: baby diaper leg elastic, diaper frontal tape, diaper standing leg cuff, diaper chassis construction, diaper core stabilization, diaper liquid transfer layer, diaper outer cover lamination, diaper elastic cuff lamination, feminine napkin core stabilization, feminine napkin adhesive strip, industrial filtration bonding, industrial filter material lamination, filter mask lamination, surgical gown lamination, surgical drape lamination, and perishable products packaging.

The adhesives described above may be applied to any substrate. Such substrates include wood, paper, cardboard, plastic, thermoplastic, rubber, metal, metal foil (such as aluminum foil and tin foil), metallized surfaces, cloth, non-wovens (particularly polypropylene spunbonded fibers or non-wovens), spunbonded fibers, cardboard, stone, plaster, glass (including silicon oxide (SiOₓ) coatings applied by evaporating silicon oxide onto a film surface), foam, rock, ceramics, films, polymer foams (such as polyurethane foam), substrates coated with inks, dyes, pigments, PVDC and the like or combinations thereof. Additional substrates include polyethylene, polypropylene, polyacrylates, acrylics, polyethylene terephthalate, or any of the polymers listed above as suitable for blends. Corona treatment, electron beam irradiation, gamma irradiation, microwave or silanization may modify any of the above substrates.

### Films

The functional polymer compositions disclosed herein may be formed into monolayer or multilayer films. These films may be formed by any of the conventional techniques known in the art including extrusion, co-extrusion, extrusion coating, lamination, blowing, tenter frame, and casting. The film may be obtained by the flat film or tubular process, which may be followed by orientation in an uniaxial direction, or in two mutually perpendicular directions in the plane of the film. One or more of the layers of the film may be oriented in the transverse and/or longitudinal directions to the same or different extents. This orientation may occur before or after the individual layers are brought together. For example a polyethylene layer can be extrusion coated or laminated onto an oriented polypropylene layer or the polyethylene and polypropylene can be coextruded together into a film then oriented. Likewise, oriented polypropylene could be laminated to oriented polyethylene or oriented polyethylene could be coated onto polypropylene then optionally the combination could be oriented even further. Typically the films are oriented in the Machine Direction (MD) at a ratio of up to 15, advantageously between 5 and 7, and in the Transverse Direction (TD) at a ratio of up to 15 advantageously 7 to 9. However, in another form, the film is oriented to the same extent in both the MD and TD directions. In another form, the layer comprising the polymer composition disclosed herein may be combined with one or more other layers. The other layer(s) may be any layer typically included in multilayer film structures. For example the other layer or layers may be:

1. Polyolefins. Polyolefins include homopolymers or copolymers of C₂ to C₄₀ olefins, alternately C₂ to C₂₀ olefins, alternately a copolymer of an α-olefin and another olefin or α-olefin (ethylene is defined to be an α-olefin for purposes of this invention). Examples include homopolyethylene, homopolypropylene, propylene copolymerized with ethylene and or butene, ethylene copolymerized with one or more of propylene, butene or hexene, and optional dienes. Examples include thermoplastic polymers such as ultra low density polyethylene, very low density polyethylene, linear low density polyethylene, low density polyethylene, medium density polyethylene, high density polyethylene, polypropylene, isotactic polypropylene, highly isotactic polypropylene, syndiotactic polypropylene, random copolymer of propylene and ethylene and/or butene and/or hexene, elastomers such as ethylene propylene rubber, ethylene propylene diene monomer rubber, neoprene, and blends of thermoplastic polymers and elastomers, such as for example, thermoplastic elastomers and rubber toughened plastics.
2. Polar polymers. Polar polymers include homopolymers and copolymers of esters, amides, acrylates, anhydrides, copolymers of a C₂ to C₂₀ olefin, such as ethylene and/or propylene and/or butene with one or more polar monomers such as acetates, anhydrides, esters, alcohol, and or acrylics. Examples include polyesters, polyamides, ethylene vinyl acetate copolymers, and polyvinyl chloride.
3. Cationic polymers. Cationic polymers include polymers or copolymers of germinally disubstituted olefins, alpha-heteroatom olefins and/or styrenic monomers. Examples of germinally disubstituted olefins include isobutylene, isopentane, isoheptane, isohexane, isooctane, isodecene, and isododecane. Examples of α-heteroatom olefins include vinyl ether and vinyl carbazole, advantageous styrenic monomers include styrene, alkyl styrene, para-alkyl styrene, alpha-methyl styrene, chloro-styrene, and bromo-para-methyl styrene. Examples of cationic polymers include butyl rubber, isobutylene copolymerized with para methyl styrene, polystyrene, and poly-α-methyl styrene.
4. Miscellaneous. Other layers can be paper, wood, cardboard, metal, metal foils (such as aluminum foil and tin foil), metallized surfaces, glass (including silicon oxide (SiOx) coatings applied by evaporating silicon oxide onto a film surface), fabric, spunbonded fibers, and non-wovens (particularly polypropylene spunbonded fibers or non-wovens), and substrates coated with inks, dyes, pigments, PVDC and the like. The films may vary in thickness depending on the intended application; however films of a thickness from 1 to 250 μm are usually suitable. Films intended for packaging are usually from 10 to 60 μm thick. The thickness of the sealing layer is typically 0.2 to 50 μm. There may be a sealing layer on both the inner and outer surfaces of the film or the sealing layer may be present on only the inner or the outer surface. Additives such as block, antiblock, antioxidants, pigments, fillers, processing aids, UV stabilizers, neutralizers, lubricants, surfactants and/or nucleating agents may also be present in one or more than one layer in the films. Useful additives include silicon dioxide, titanium dioxide, polydimethylsiloxane, talc, dyes, wax, calcium stearate, carbon black, low molecular weight resins and glass beads. In another form, one or more layers may be modified by corona treatment, electron beam irradiation, gamma irradiation, or microwave. In some forms, one or both of the surface layers is modified by corona treatment. The films described herein may also comprise from 5 to 60 weight %, based upon the weight of the polymer and the resin, of a hydrocarbon resin. The resin may be combined with the polymer of the seal layer(s) or may be combined with the polymer in the core layer(s). The resin advantageously has a softening point above 100° C., even more advantageously from 130 to 180° C. Useful hydrocarbon resins include those described above. The films comprising a hydrocarbon resin may be oriented in uniaxial or biaxial directions to the same or different degrees.

The films described above may be used as stretch and/or cling films. Stretch/cling films are used in various bundling, packaging and palletizing operations. To impart cling properties to, or improve the cling properties of, a particular film, a number of well-known tackifying additives have been utilized. Common tackifying additives include polybutenes, terpene resins, alkali metal stearates and hydrogenated rosins and rosin esters. The well-known physical process referred to as corona discharge can also modify the cling properties of a film. Some polymers (such as ethylene methyl acrylate copolymers) do not need cling additives and can be used as cling layers without tackifiers. Stretch/clings films may comprise a slip layer comprising any suitable polyolefin or combination of polyolefins such as polyethylene, polypropylene, copolymers of ethylene and propylene, and polymers obtained from ethylene and/or propylene copolymerized with minor amounts of other olefins, particularly C₄-C₁₂ olefins. Particularly useful are polypropylene and linear low density polyethylene (LLDPE). Suitable polypropylene is normally solid and isotactic, i.e., greater than 90% hot heptane insolubles, having wide ranging melt flow rates of from about 0.1 to about 300 g/10 min. Additionally, the slip layer may include one or more anti-cling (slip and/or antiblock) additives, which may be added during the production of the polyolefin or subsequently blended in to improve the slip properties of this layer. Such additives are well-known in the art and include, for example, silicas, silicates, diatomaceous earths, talcs and various lubricants. These additives are advantageously utilized in amounts ranging from about 100 ppm to about 20,000 ppm, more advantageously between about 500 ppm to about 10,000 ppm, by weight based upon the weight of the slip layer. The slip layer may, if desired, also include one or more other additives as described above.

Polymers produced herein can be used for nonwovens, sealing layers, oriented polypropylene, and high-clarity thermoforming.

The films produced herein may also be used in heat sealing applications, particularly as heat sealing layers e.g. surface layers, in multilayer films. In one form, the polymers produced herein are used in heat sealing applications, such as packaging, form, fill and seal applications and packaging films such as biaxially oriented films. The polymers produced herein, alone or blended with other polymers, may be coextruded or laminated onto another polymer (typically in a film structure) and used in applications requiring good heat sealing.

### Melt-Blown and Spun-Bond Fabrics

The functional polymer compositions disclosed herein may be useful for melt blown and spunbond fabrics. More particularly, the functional polymer compositions disclosed herein may be used for making PP for spunbonded (SB) and melt blown (MB) fibers. Typical invention polymers have ash levels below 1000, 900, 700, 500, 400, 300, 200, 100, 50, 10, 1, 0.5, or 0.1 ppm. Some embodiments have ash levels of 1-500 ppb. All these characteristics combine to reduce polymer build-up on the die exits. These polymer blend products can have high MFRs from 300-5000 useful for fiber applications.

### Waxes

An appropriate choice of operating conditions and monomer and comonomer feeds may yield blends of polypropylene waxes from the functional polymer compositions disclosed herein. Some forms are blended isotactic polypropylene waxes. As such these materials are well suited for viscosity modification in adhesives, as carriers for inks, and other applications. Some polypropylene waxes have melt viscosities of from 3-2000 cP at 180° C. Some forms produce syndiotactic polypropylene waxes.

### Other Articles

Laminates comprising the functional polymer compositions disclosed herein can be used as a thermoformable sheet where the substrate is either sprayed or injection molded to couple it with the ionomer/tie-layer laminate sheet. The composite is formed into the desired shape to make the article, or composite article. Various types of substrate materials form highly desirable articles. The laminate can be used with plastic substrates such as homopolymers, copolymers, foams, impact copolymers, random copolymers, and other applications. Specifically, some articles in which the polymers disclosed herein can be incorporated are the following: vehicle parts, especially exterior parts such as bumpers and grills, rocker panels, fenders, doors, hoods, trim, and other parts can be made from the laminates, composites and methods of the invention.

Other articles can also be named, for example: counter tops, laminated surface counter tops, pool liners/covers/boat covers, boat sails, cable jacketing, motorcycles/snowmobiles/outdoor vehicles, marine boat hulls/canoe interior and exterior, luggage, clothing/fabric (combined with non-wovens), tent material, GORETEX™, gamma-radiation resistant applications, electronics housing (TV's, VCR's and computers), a wood replacement for decks and other outdoor building materials, prefab buildings, synthetic marble panels for construction, wall covering, hopper cars, floor coating, polymer/wood composites, vinyl tile, bath/shower/toilet applications and translucent glass replacement, sidings, lawn/outdoor furniture, appliances such as refrigerators, washing machines, etc., children's toys, reflective signage and other reflective articles on roads and clothing, sporting equipment such as snowboards, surfboards, skis, scooters, wheels on in-line skates, CD's for scratch resistance, stadium seats, aerospace reentry shields, plastic paper goods, sports helmets, plastic microwaveable cookware, and other applications for coating plastics and metal where a highly glossy and scratch resistant surface is desirable, while not being subject to algae/discoloration.

The polypropylene copolymer functional polymer compositions disclosed herein are suitable for applications such as molded articles, including injection and blow molded bottles and molded items used in automotive articles, such as automotive interior and exterior trims. Examples of other methods and applications for making polypropylene polymers and for which polypropylene polymers may be useful are described in the ENCYCLOPEDIA OF CHEMICAL TECHNOLOGY, by Kirk-Othmer, Fourth Edition, vol. 17, at pages 748-819, which are incorporated by reference herein. In those instances where the application is for molded articles, the molded articles may include a variety of molded parts, particularly molded parts related to and used in the automotive industry such as, for example, bumpers, side panels, floor mats, dashboards and instrument panels. Foamed articles are another application and examples where foamed plastics, such as foamed polypropylene, are useful may be found in ENCYCLOPEDIA OF CHEMICAL TECHNOLOGY, by Kirk-Othmer, Fourth Edition, vol. 11, at pages 730-783, which are incorporated by reference herein. Foamed articles are particularly useful for construction and automotive applications. Examples of construction applications include heat and sound insulation, industrial and home appliances, and packaging. Examples of automotive applications include interior and exterior automotive parts, such as bumper guards, dashboards and interior liners.

The functional polymer compositions disclosed herein may be suitable for such articles as automotive components, wire and cable jacketing, pipes, agricultural films, geomembranes, toys, sporting equipment, medical devices, casting and blowing of packaging films, extrusion of tubing, pipes and profiles, sporting equipment, outdoor furniture (e.g., garden furniture) and playground equipment, boat and water craft components, and other such articles. In particular, the compositions are suitable for automotive components such as bumpers, grills, trim parts, dashboards and instrument panels, exterior door and hood components, spoiler, wind screen, hub caps, mirror housing, body panel, protective side molding, and other interior and external components associated with automobiles, trucks, boats, and other vehicles.

Other useful articles and goods may be formed economically, including: crates, containers, packaging, labware, such as roller bottles for culture growth and media bottles, office floor mats, instrumentation sample holders and sample windows; liquid storage containers such as bags, pouches, and bottles for storage and IV infusion of blood or solutions; packaging material including those for any medical device or drugs including unit-dose or other blister or bubble pack as well as for wrapping or containing food preserved by irradiation. Other useful items include medical tubing and valves for any medical device including infusion kits, catheters, and respiratory therapy, as well as packaging materials for medical devices or food which is irradiated including trays, as well as stored liquid, particularly water, milk, or juice, containers including unit servings and bulk storage containers as well as transfer means such as tubing, pipes, and such.

### Molded Products

The functional polymer compositions disclosed herein may also be used to prepare the molded products in any molding process, including but not limited to, injection molding, gas-assisted injection molding, extrusion blow molding, injection blow molding, injection stretch blow molding, compression molding, rotational molding, foam molding, thermoforming, sheet extrusion, and profile extrusion. The molding processes are well known to those of ordinary skill in the art.

The functional polymer compositions disclosed herein may be shaped into desirable end use articles by any suitable means known in the art. Thermoforming, vacuum forming, blow molding, rotational molding, slush molding, transfer molding, wet lay-up or contact molding, cast molding, cold forming matched-die molding, injection molding, spray techniques, profile co-extrusion, or combinations thereof are typically used methods.

Thermoforming is a process of forming at least one pliable plastic sheet into a desired shape. An embodiment of a thermoforming sequence is described, however this should not be construed as limiting the thermoforming methods useful with the compositions of this invention. First, an extrudate film of the composition of this invention (and any other layers or materials) is placed on a shuttle rack to hold it during heating. The shuttle rack indexes into the oven which pre-heats the film before forming. Once the film is heated, the shuttle rack indexes back to the forming tool. The film is then vacuumed onto the forming tool to hold it in place and the forming tool is closed. The forming tool can be either “male” or “female” type tools. The tool stays closed to cool the film and the tool is then opened. The shaped laminate is then removed from the tool.

Thermoforming is accomplished by vacuum, positive air pressure, plug-assisted vacuum forming, or combinations and variations of these, once the sheet of material reaches thermoforming temperatures, typically of from 140° C. to 185° C. or higher. A pre-stretched bubble step is used, especially on large parts, to improve material distribution. In one form, an articulating rack lifts the heated laminate towards a male forming tool, assisted by the application of a vacuum from orifices in the male forming tool. Once the laminate is firmly formed about the male forming tool, the thermoformed shaped laminate is then cooled, typically by blowers. Plug-assisted forming is generally used for small, deep drawn parts. Plug material, design, and timing can be critical to optimization of the process. Plugs made from insulating foam avoid premature quenching of the plastic. The plug shape is usually similar to the mold cavity, but smaller and without part detail. A round plug bottom will usually promote even material distribution and uniform side-wall thickness. For a semicrystalline polymer such as polypropylene, fast plug speeds generally provide the best material distribution in the part.

The shaped laminate is then cooled in the mold. Sufficient cooling to maintain a mold temperature of 30° C. to 65° C. is desirable. The part is below 90° C. to 100° C. before ejection in one embodiment. For good behavior in thermoforming, the lowest melt flow rate polymers are desirable. The shaped laminate is then trimmed of excess laminate material.

Blow molding is another suitable forming means, which includes injection blow molding, multi-layer blow molding, extrusion blow molding, and stretch blow molding, and is especially suitable for substantially closed or hollow objects, such as, for example, gas tanks and other fluid containers. Blow molding is described in more detail in, for example, CONCISE ENCYCLOPEDIA OF POLYMER SCIENCE AND ENGINEERING 90-92 (Jacqueline I. Kroschwitz, ed., John Wiley & Sons 1990).

In yet another form of the formation and shaping process, profile co-extrusion can be used. The profile co-extrusion process parameters are as above for the blow molding process, except the die temperatures (dual zone top and bottom) range from 150° C.-235° C., the feed blocks are from 90° C.-250° C., and the water cooling tank temperatures are from 10° C.-40° C.

One embodiment of an injection molding process is described as follows. The shaped laminate is placed into the injection molding tool. The mold is closed and the substrate material is injected into the mold. The substrate material has a melt temperature between 200° C. and 300° C. in one embodiment, and from 215° C. and 250° C. and is injected into the mold at an injection speed of between 2 and 10 seconds. After injection, the material is packed or held at a predetermined time and pressure to make the part dimensionally and aesthetically correct. Typical time periods are from 5 to 25 seconds and pressures from 1,380 kPa to 10,400 kPa. The mold is cooled between 10° C. and 70° C. to cool the substrate. The temperature will depend on the desired gloss and appearance desired. Typical cooling time is from 10 to 30 seconds, depending on part on the thickness. Finally, the mold is opened and the shaped composite article ejected.

Likewise, molded articles may be fabricated by injecting molten polymer into a mold that shapes and solidifies the molten polymer into desirable geometry and thickness of molded articles. Sheet may be made either by extruding a substantially flat profile from a die, onto a chill roll, or alternatively by calendaring. Sheet will generally be considered to have a thickness of from 10 mils to 100 mils (254 μm to 2540 μm), although sheet may be substantially thicker. Tubing or pipe may be obtained by profile extrusion for uses in medical, potable water, land drainage applications or the like. The profile extrusion process involves the extrusion of molten polymer through a die. The extruded tubing or pipe is then solidified by chill water or cooling air into a continuous extruded articles. The tubing will generally be in the range of from 0.31 cm to 2.54 cm in outside diameter, and have a wall thickness of in the range of from 254 μm to 0.5 cm. The pipe will generally be in the range of from 2.54 cm to 254 cm in outside diameter, and have a wall thickness of in the range of from 0.5 cm to 15 cm. Sheet made from the products of an embodiment of a version of the present invention may be used to form containers. Such containers may be formed by thermoforming, solid phase pressure forming, stamping and other shaping techniques. Sheets may also be formed to cover floors or walls or other surfaces.

In one form of the thermoforming process, the oven temperature is between 160° C. and 195° C., the time in the oven between 10 and 20 seconds, and the die temperature, typically a male die, between 10° C. and 71° C. The final thickness of the cooled (room temperature), shaped laminate is from 10 g/m to 6000 μm in one embodiment, from 200 μm to 6000 μm in another embodiment, and from 250 μm to 3000 μm in yet another embodiment, and from 500 μm to 1550 μm in yet another embodiment, a desirable range being any combination of any upper thickness limit with any lower thickness limit.

In one form of the injection molding process, wherein a substrate material in injection molded into a tool including the shaped laminate, the melt temperature of the substrate material is between 230° C. and 255° C. in one embodiment, and between 235° C. and 250° C. in another embodiment, the fill time from 2 to 10 seconds in one embodiment, from 2 to 8 seconds in another embodiment, and a tool temperature of from 25° C. to 65° C. in one embodiment, and from 27° C. and 60° C. in another embodiment. In a desirable embodiment, the substrate material is at a temperature that is hot enough to melt any tie-layer material or backing layer to achieve adhesion between the layers.

In yet another form, the functional polymer, compositions disclosed herein may be secured to a substrate material using a blow molding operation. Blow molding is particularly useful in such applications as for making closed articles such as fuel tanks and other fluid containers, playground equipment, outdoor furniture and small enclosed structures. In one form, compositions are extruded through a multi-layer head, followed by placement of the uncooled laminate into a parison in the mold. The mold, with either male or female patterns inside, is then closed and air is blown into the mold to form the part.

It will be understood by those skilled in the art that the steps outlined above may be varied, depending upon the desired result. For example, an extruded sheet of the blend compositions disclosed herein may be directly thermoformed or blow molded without cooling, thus skipping a cooling step. Other parameters may be varied as well in order to achieve a finished composite article having desirable features.

### Non-Wovens and Fibers

The functional polymer compositions disclosed herein may be used to prepare nonwoven fabrics and fibers in any nonwoven fabric and fiber making process, including but not limited to, melt blowing, spunbonding, film aperturing, and staple fiber carding. A continuous filament process may also be used. One example is a spunbonding process. The spunbonding process is well known in the art. Generally it involves the extrusion of fibers through a spinneret. These fibers are then drawn using high velocity air and laid on an endless belt. A calendar roll is generally then used to heat the web and bond the fibers to one another although other techniques may be used such as sonic bonding and adhesive bonding. The fabric may be prepared with mixed metallocene polypropylene alone, physically blended with other mixed metallocene polypropylene or physically blended with single metallocene polypropylene. Likewise the fabrics of this invention may be prepared with mixed metallocene polypropylene physically blended with conventional Ziegler-Natta produced polymer. If blended, the fabric of this invention is advantageously comprised of at least 50% mixed metallocene polypropylene. With these nonwoven fabrics, manufacturers can maintain the desirable properties of fabrics prepared with metallocene produced polypropylene while increasing fabric strength and potentially increased line speed compared to fabrics made using conventional polymers.

### A. Fiber Preparation:

The formation of woven and nonwoven articles typically requires the manufacture of fibers by extrusion followed by weaving or bonding. The extrusion process is typically accompanied by mechanical or aerodynamic drawing of the fibers. Essentially all fibers are oriented both during the extrusion process as well as during the process of manufacture of the non woven article.

The three more conventional fiber operations, continuous filament, bulked continuous filament, and staple, are useful as means for preparing fibers from the polymer compositions disclosed herein. Typically the molten blend is extruded through the holes in a die (spinneret) between 0.3 mm to 0.8 mm (10 mil to 30 mil) in diameter. Low melt viscosity of the polymer-blend is advantageous and is typically achieved through the use of high melt temperature (230° C. to 280° C.) and high melt flow rates (15 g/10 min to 40 g/10 min). A relatively large extruder is typically equipped with a manifold to distribute a high output of molten blend to a bank of eight to twenty spinnerets. Each spinhead is typically equipped with a separate gear pump to regulate output through that spinhead; a filter pack, supported by a “breaker plate”; and the spinneret plate within the head. The number of holes in the spinneret plate determines the number of filaments in a yarn and varies considerably with the different yarn constructions, but it is typically in the range of 50 to 250. The holes are typically grouped into round, annular, or rectangular patterns to assist in good distribution of the quench air flow.

Continuous filament yarns typically range from 40 denier to 2,000 denier (denier=number of grams/9000 yd). Filaments typically range from 1 to 20 dpf, but can be larger. Spinning speeds are typically 800 m/min to 1500 m/min (2500 μl/min to 5000 ft/min). The filaments are drawn at draw ratios of 3:1 or more (one- or two-stage draw) and wound onto a package. Two-stage drawing allows higher draw ratios to be achieved. Winding speeds are 2,000 m/min to 3,500 n/min (6,600 ft/min to 11,500 ft/min). Spinning speeds in excess of 900 m/min (3000 ft/min) require a NMWD to get the best spinnability with the finer filaments.

### B. Bulked Continuous Filament Formation:

Bulked Continuous Filament fabrication processes fall into two basic types, one-step and two step. In the older, two-step process, an undrawn yarn is spun at less than 1,000 m/min (3,300 ft/min), usually 750 m/min, and placed on a package. The yarn is drawn (usually in two stages) and “bulked” on a machine called a texturiser. Winding and drawing speeds are limited by the bulking or texturizing device to 2,500 m/min (8,200 ft/min) or less. Typically if secondary crystallization occurs in the two-step CF process, then one typically promptly uses draw texturizing. The most common process today is the one-step spin/draw/text (SDT) process. This process provides better economics, efficiency and quality than the two-step process. It is similar to the one-step CF process, except that the bulking device is in-line. Bulk or texture changes yarn appearance, separating filaments and adding enough gentle bends and folds to make the yarn appear fatter (bulkier).

### C. Staple Fiber Formation:

There are two basic staple fiber fabrication processes: traditional and compact spinning. The traditional process involves two steps: 1) producing, applying finish, and winding followed by 2) drawing, a secondary finish application, crimping, and cutting into staple. Filaments can range from 1.5 dpf to >70 dpf, depending on the application. Staple length can be as short as 7 mm or as long as 200 mm (0.25 in. to 8 in.) to suit the application. For many applications the fibers are crimped. Crimping is accomplished by over-feeding the tow into a steam-heated stuffer box with a pair of nip rolls. The over-feed folds the tow in the box, forming bends or crimps in the filaments. These bends are heat-set by steam injected into the box.

### D. Melt-Blown Fibers:

Melt blown fibers can make very fine filaments and produce very lightweight fabrics with excellent uniformity. The result is often a soft fabric with excellent “barrier” properties. In the melt blown process molten polymer moves from the extruder to the special melt blowing die. As the molten filaments exit the die, they are contacted by high temperature, high velocity air (called process or primary air). This air rapidly draws and, in combination with the quench air, solidifies the filaments. The entire fiber forming process generally takes place within 7 mm (0.25 in.) of the die. The fabric is formed by blowing the filaments directly onto a forming wire, 200 mm to 400 mm (8 in. to 15 in.) from the spinnerets.

Melt blown microfibers useful in the present invention can be prepared as described in Van A. Wente, “Superfine Thermoplastic Fibers,” Industrial Engineering Chemistry, vol. 48, pp. 1342-1346 and in Report No. 4364 of the Naval Research Laboratories, published May 25, 1954, entitled “Manufacture of Super Fine Organic Fibers” by Van A. Wente et al. In some advantageous embodiments, the microfibers are used in filters. Such blown microfibers typically have an effective fiber diameter of from about 3 to 30 micrometers advantageously from about 7 to 15 micrometers, as calculated according to the method set forth in Davies, C. N., “The Separation of Airborne Dust and Particles”, Institution of Mechanical Engineers, London, Proceedings 1B, 1952.

### E. Spun-bonded Fibers:

Fiber formation may also be accomplished by extrusion of the molten polymer from either a large spinneret having several thousand holes or with banks of smaller spinnerets containing as few as 40 holes. After exiting the spinneret, the molten fibers are quenched by a cross-flow air quench system, then pulled away from the spinneret and attenuated (drawn) by high pressure air. There are two methods of air attenuation, both of which use the venturi effect. The first draws the filament using an aspirator slot (slot draw), which runs the width of the machine. The second method draws the filaments through a nozzle or aspirator gun. Filaments formed in this manner are collected on a screen (“wire”) or porous forming belt to form the fabric. The fabric is then passed through compression rolls and then between heated calendar rolls where the raised lands on one roll bond the fabric at points covering 20% to 40% of its area.

### F. Annealing:

In additional embodiments, the mechanical properties of fibers comprising the polymer compositions disclosed herein may be improved by the annealing the fibers or the non-woven materials made from the blends of this invention. Annealing is often combined with mechanical orientation, although annealing is advantageous. Annealing partially relieves the internal stress in the stretched fiber and restores the elastic recovery properties of the blend in the fiber. Annealing has been shown to lead to significant changes in the internal organization of the crystalline structure and the relative ordering of the amorphous and semicrystalline phases. Annealing typically leads to improved elastic properties. The fiber or fabric is advantageously annealed at a temperature of at least 40° F., advantageously at least 20° F. above room temperature (but slightly below the crystalline melting point of the blend). Thermal annealing of the blend is conducted by maintaining the polymer blends or the articles made from a such a blend at temperature between room temperature to a maximum of 160° C. or more advantageously to a maximum of 130° C. for a period between 5 minutes to less than 7 days. A typical annealing period is 3 days at 50° C. or 5 minutes at 100° C. While the annealing is done in the absence of mechanical orientation, the latter can be a part of the annealing process on the fiber (past the extrusion operation). Mechanical orientation can be done by the temporary, forced extension of the fiber for a short period of time before it is allowed to relax in the absence of the extensional forces. Oriented fibers are conducted by maintaining the fibers or the articles made from a blend at an extension of 100% to 700% for a period of 0.1 seconds to 24 hours. A typical orientation is an extension of 200% for a momentary period at room temperature.

For orientation, a fiber at an elevated temperature (but below the crystalline melting point of the polymer) is passed from a feed roll of fiber around two rollers driven at different surface speeds and finally to a take-up roller. The driven roller closest to the take-up roll is driven faster than the driven roller closest to the feed roll, such that the fiber is stretched between the driven rollers. The assembly may include a roller intermediate the second roller and take-up roller to cool the fiber. The second roller and the take-up roller may be driven at the same peripheral speeds to maintain the fiber in the stretched condition. If supplementary cooling is not used, the fiber will cool to ambient temperature on the take up roll. For more information on fiber and non-woven production, see POLYPROPYLENE HANDBOOK, E. P. Moore, Jr., et al., Hanser/Gardner Publications, Inc. New York, 1996, pages 314 to 322, which is incorporated by reference herein.

### G. Nonwoven Web:

In one embodiment, a nonwoven fiber web is prepared from the polymer compositions disclosed herein, in particular a polymer/NFP blend made by the processes disclosed herein. The fibers employed in such a web typically have denier ranging from about 0.5 to about 10 (about 0.06 to about 11 tex), although higher denier fibers may also be employed. Fibers having denier from about 0.5 to 3 (0.06 to about 3.33 tex) are particularly advantageous. (“Denier” means weight in grams of 9000 meters of fiber, whereas “tex” means weight in grams per kilometer of fiber.) Fiber stock having a length ranging from about 0.5 to about 10 cm may be employed as a starting material, and particularly fiber lengths ranging from about 3 to about 8 cm. Nonwoven webs of fibers may be made using methods well documented in the nonwoven literature (see for example Turbak, A. “Nonwovens: An Advanced Tutorial”, Tappi Press, Atlanta, Ga., (1989). The uncoated (i.e., before application of any binder) web should have a thickness in the range of about 10 to 100 mils (0.254 to 2.54 mm), or 30 to 70 mils (0.762 to 1.778 mm), or 40 to 60 mils (1.02 to 1.524 mm). These thicknesses may be achieved either by the carding/crosslapping operation or via fiber entanglement (e.g., hydroentanglement, needling, and the like). The basis weight of the uncoated web advantageously ranges from about 50 g/m² up to about 250 g/m². In some embodiments, one may improve the tensile and tear strength of the inventive articles, and reduce lint on the surface of the articles, by entangling (such as by needletacking, hydroentanglement, and the like) the nonwoven web, or calendering the uncoated and/or coated and cured nonwoven web. Hydroentanglement may be employed in cases where fibers are water insoluble. Calendering of the nonwoven web at temperatures from about 5 to about 40° C. below the melting point of the fiber may reduce the likelihood of lint attaching to the surface of the ultimate articles and provide a smooth surface. Embossing of a textured pattern onto the nonwoven web may be performed simultaneously with calendering, or in a subsequent step. In addition to the polyolefins and the NFP's of this invention, it may also be desirable to add colorants (especially pigments), softeners (such as ethers and alcohols), fragrances, fillers (such as for example silica, alumina, and titanium dioxide particles), and bactericidal agents (for example iodine, quaternary ammonium salts, and the like) to the blends.

Likewise the nonwoven webs and fibers may be coated with other materials, such as binders, adhesives, reflectants, and the like. Coating of the nonwoven web or the fiber may be accomplished by methods known in the art, including roll coating, spray coating, immersion coating, gravure coating, or transfer coating. The coating weight as a percentage of the total wiping article may be from about 1% to about 95%, or from about 10% to about 60%, or 20 to 40%.

Staple fibers may also be present in the nonwoven web. The presence of staple fibers generally provides a more lofty, less dense web than a web of only blown microfibers. No more than about 90 weight percent staple fibers are present, or alternately no more than about 70 weight percent. Such webs containing staple fiber are disclosed in U.S. Pat. No. 4,118,531 (Hauser) which is incorporated herein by reference.

Sorbent particulate material such as activated carbon or alumina may also be included in the web. Such particles may be present in amounts up to about 80 volume percent of the contents of the web. Such particle-loaded webs are described, for example, in U.S. Pat. No. 3,971,373 (Braun), U.S. Pat. No. 4,100,324 (Anderson) and U.S. Pat. No. 4,429,001 (Kolpin et al.), which are incorporated herein by reference.

The fibers and nonwoven webs prepared using the functional polymer compositions disclosed herein may be formed into fabrics, garments, clothing, medical garments, surgical gowns, surgical drapes, diapers, training pants, sanitary napkins, panty liners, incontinent wear, bed pads, bags, packaging material, packages, swimwear, body fluid impermeable backsheets, body fluid impermeable layers, body fluid permeable layers, body fluid permeable covers, absorbents, tissues, nonwoven composites, liners, cloth linings, scrubbing pads, face masks, respirators, air filters, vacuum bags, oil and chemical spill sorbents, thermal insulation, first aid dressings, medical wraps, fiberfill, outerwear, bed quilt stuffing, furniture padding, filter media, scrubbing pads, wipe materials, hosiery, automotive seats, upholstered furniture, carpets, carpet backing, filter media, disposable wipes, diaper coverstock, gardening fabric, geomembranes, geotextiles, sacks, housewrap, vapor barriers, breathable clothing, envelops, tamper evident fabrics, protective packaging, and coasters.

The fibers prepared using the functional polymer compositions disclosed herein may be formed into yarns, woven fabrics, nonwoven fabrics, hook and loop fasteners, fabrics, garments, clothing, medical garments, surgical gowns, surgical drapes, diapers, training pants, sanitary napkins, panty liners, incontinent wear, bed pads, bags, packaging material, packages, swimwear, body fluid impermeable backsheets, body fluid impermeable layers, body fluid permeable layers, body fluid permeable covers, absorbents, tissues, nonwoven composites, liners, cloth linings, scrubbing pads, face masks, respirators, air filters, vacuum bags, oil and chemical spill sorbents, thermal insulation, first aid dressings, medical wraps, fiberfill, outerwear, bed quilt stuffing, furniture padding, filter media, scrubbing pads, wipe materials, hosiery, automotive seats, upholstered furniture, carpets, carpet backing, filter media, disposable wipes, diaper coverstock, gardening fabric, geomembranes, geotextiles, sacks, housewrap, vapor barriers, breathable clothing, envelops, tamper evident fabrics, protective packaging, and coasters.

The following examples illustrate the present disclosure and the advantages thereto without limiting the scope thereof.

### Test Methods

All viscosities of fluids disclosed herein were measured at 100° C. according to ASTM 445 method.

AATCC (American Association of Textile Chemists and Colorists, Research Triangle Park, N.C.) test method 147 provides an antibacterial assessment of textile materials. Testing was conducted by Biosan Laboratories, Inc., Warren, Mich. The parallel streak method was strictly followed, and is a qualitative screening test to demonstrate bacteriostatic (inhibition of multiplication and growth) activity by diffusion of the antimicrobial agent through agar. The test sample (textile) was placed in intimate contact with a nutrient agar surface which has been previously streaked (“parallel streaks”) with an inoculum of test organism, e.g. Staphylococcus aureus. After 24 hours incubation, the bacteriostatic activity was demonstrated by a clear area of interrupted growth underneath and along the sides of the test material.

For purpose of this disclosure, unless otherwise noted, physical and chemical properties of the fluids described were measured using the following test methods.

Fluid Properties

| | |
|---|---|
| Kinematic Viscosity (KV) | ASTM D 445 |
| Viscosity Index (VI) | ASTM D 2270 |
| Pour Point | ASTM D 97 |
| Specific Gravity and Density | ASTM D 4052 (15.6/15.6° C.) |
| Flash Point | ASTM D 92 |
| Noack volatility (evaporative loss) | ASTM D 5800 |
| Glass Transition Temperature (T_{g}) | ASTM 1356 |
| Branch Paraffin: N-paraffin ratio | ¹³C-NMR |
| % mono-methyl species | ¹³C-NMR |
| % side chains with X number | ¹³C-NMR |
| of carbons | |
| Saturates Content | ASTM D 2007 |
| Sulfur Content | ASTM D 2622 |
| Nitrogen Content | ASTM D 4629 |
| Aniline Point | ASTM D 611 |

The sample molecular weight can be determined by Gel Permeation Chromatography (GPC) method using polystyrene as a calibration standard. Mₙ, M_{w} and molecular weight distribution values were reported. Suitable GPC methods involving a DRI detector are generally described in “Modern Size Exclusion Liquid Chromatographs”, W. W. Yan, J. J. Kirkland, and D. D. Bly (J. Wiley & Sons, 1979).

### EXAMPLES

### Example 1

### Antibacterial Polypropylene Test

To simulate the effect of a real fabric, a polypropylene strip was prepared and used in the test. The components of the test strip included a polypropylene with 3 wt % ethylene, ˜35 MFR. The anti-bacterial agent used was Irgansan available from Aldrich Chemical Co., which is a solid at room temperature with a melting point of 57° C. and a boiling point of 120° C. Triclosan has the following structure:

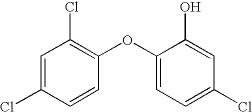

The fluid used was a PAO/ester, which is a synthetic fluid used as a delivery/carrier fluid. Because Triclosan has low solubility in PAO alone, 5% Esterex A51 available from ExxonMobil Chemical Co., Houston, Tex. was added to solubilize the Tricolsan.

The procedures for preparation of PP strips were as follows. Six triclosan stock solutions were prepared by mixing 5 grams of triclosan, 5 grams of Esterex A51 (tridecyl adipate or MCP121) and 90 grams of PAO with different viscosity grades. The solution data are summarized in Table 2 below.

TABLE 2
PAO-Triclosan Stock Solution:

| Notebook No. | | | | | | | |
|---|---|---|---|---|---|---|---|
| 24892-63 | 1 | 2 | 3 | 4 | 5 | 6 | 7 |
| PAO | 6 | 40 | 100 | 300 | Cascade | Cascade | Cascade |
| | | | | | PAO-1 | PAO-2 | PAO-3 |
| Triclosan | 5 | 5 | 5 | 5 | 5 | 5 | 5 |
| (Irgasan), wt % | | | | | | | |
| PAO, wt % | 90 | 90 | 90 | 90 | 90 | 90 | 90 |
| MCP 121 ester, | 5 | 5 | 5 | 5 | 5 | 5 | 5 |
| wt % | | | | | | | |
| Total solution wt. | 200 | 200 | 200 | 200 | 200 | 200 | 200 |
| gram | | | | | | | |
| Triclosan, gram | 10 | 10 | 10 | 10 | 10 | 10 | 10 |
| PAO, gram | 180 | 180 | 180 | 180 | 180 | 180 | 180 |
| MCP 121 ester, | 10 | 10 | 10 | 10 | 10 | 10 | 10 |
| gram | | | | | | | |
| 100° C. Kv, cS | 5.52 | 31.63 | 77.71 | 209.34 | 40.39 | 18.97 | 28.48 |
| 40° C. kV, cS | 29.44 | 315.04 | 944.85 | 2060.45 | 349.42 | 150.74 | 261.49 |
| VI | 127 | 139 | 159 | 234 | 168 | 133 | 135 |

Cascade PAO-1 = 50 g each PAO 6, 40, 100 and 300
Cascade PAO-2 = 50 g each PAO 6, 10, 40 and 100
Cascade PAO-3 = 67.7 g each PAO 10, 40 and 100 (vis = 32 cS)

Preparation of sample 24892-114-0 to 7: The polypropylene described above (85% by weight) was melt blended in a mixer with 15 wt % triclosan stock solution as prepared in Table 2. The mixed polymer was then compression molded into 4″×4″ squares with less than 1 mm thickness. Two methods were used to prepare the PP strip with very different results. One method includes fast cooling after the compression molding (quench sample with a 7° C. cooling surface). The sample strip had PAO fluid on surface indicating fast PAO migration after a few hours at room temperature. The other method included slow cooling after the compression molding (cool sample in ambient air, 23° C.). Sample strip appeared very dry with no apparent PAO fluid on surface. When this sample was put into 70° C. oven, slight oiliness was observed on the surface.

Three fast cooling PP strips and three slow cooling PP strips prepared using the above methods were sent to Biosan of Warren, Mich. for anti-bacterial activity testing. The test results are summarized in Table 3 below. Preparation of sample 24892-131-C: 2.5 gram of an ester Esterex A51 and 2.5 gram of Irgasan were mixed together. This solution was then dissolved in 50 ml of ethanol. 5 gram of a MCM41 powder was added to this ethanol solution and stirred for one hour at 25° C. The mixture was then stirred in open air for 16 hours while ethanol slowly evaporated. The remaining dry powder was further dried in a vacuum at 50° C. for five hours. 0.45 grams of the dry powder was mixed with 45 grams of the polypropylene polymer as described earlier and melt blended at 200° C. for 3 minutes. The resulting polymer was then compression molded to give a strip of 0.25 inches thick. A strip of molded product was used for ACCCT147 test.

Preparation of sample 24892-131-D: The same as in sample 131-C, except no ester was used, but same weight of PAO was used in place of ester.

TABLE 3
Antibacterial Test (AATCC 147 Test) of PAO-Triclosan Modified PP:

| | Sample Description | | Staphylococcus aureus | | Klebsiella pneumoniae | |
|---|---|---|---|---|---|---|
| | Wt % | | Growth | | | |
| | Triclosan | PP strip | under | | | |
| Sample | In PP strip | Preparation | sample | Zone of Inhibition | Growth under | Zone of |
| Name | (PAO type) | method | Rating (a) | in mm (b) | Sample Rating (a) | Inhibition in mm (b) |
| 27892-114-0 | 0 | Fast Cooling | 4 | NA | 4 | NA |
| | (PAO40) | | | | | |
| 24892-114-1 | 0.75 | Fast Cooling | 0 | Z = 6 | 0 | Z = 6 |
| | (PAO6) | | | | | |
| 24892-114-2 | 0.75 | Fast Cooling | 0 | Z = 6 | 0 | Z = 6 |
| | (PAO40) | | | | | |
| 24892-115-3 | 0.75 | Slow Cooling | 0 | Z = 5 | 0 | Z = 6 |
| | (PAO100) | | | | | |
| 24892-115-5 | 0.75 (mix | Slow Cooling | 0 | Z = 7 | 0 | Z = 5 |
| | PAO40) | | | | | |
| 24892-115-7 | 0.75 (mix | Slow Cooling | 0 | Z = 8 | 0 | Z = 5 |
| | PAO30) | | | | | |
| 24892-131C | 0.3 (in | Melt-pressed | 0 | Z = 5 | 0 | Z = 1 |
| | ester/M41) | | | | | |
| 24892-131D | 0.3 (in | Melt-pressed | 0 | Z = 6 | 0 | Z = 2 |
| | PAO/M41) | | | | | |
| 24892-131E | 0.2 gram AB | Melt-pressed | 1 | NA | 1 | NA |
| | polyester | | | | | |
| | fiber | | | | | |
| Positive | Positive | Positive | 0 | Z = 4 | 0 | Z = 1 |
| Control- | Control | Control | | | | |
| Glass Fiber | | | | | | |
| Filter Coated | | | | | | |
| with a | | | | | | |
| Quaternary | | | | | | |
| Compound | | | | | | |
| Negative | Negative | Negative | 4 | NA | 4 | NA |
| Control- | Control | Control | | | | |
| Glass | | | | | | |
| Fiber Filter | | | | | | |

| | |
|---|---|
| (a) Bacterial Inhibition/Growth Rating Legend | |
| No Growth underneath sample | 0 |
| Traces of growth underneath sample (less | 1 |
| than 10%) | |
| Light growth underneath sample (10-30%) | 2 |
| Medium growth underneath sample (30-60%) | 3 |
| Heavy growth underneath sample (60%-100%) | 4 |

(b) Zone of inhibition (Z1): inhibition of growth as a result of
An antimicrobial agent from the test specimen. Z1 is
expressed in average millimeter of inhibition zones around the specimen

Sample name 27892-114-0 is the control and showed significant bacteria growth. Sample names 24892-114-1, 24892-114-2, 24892-115-3, 24892-115-5, 24892-115-7, 24892-131C, and 24892-131D were prepared according to the compositions disclosed herein, and showed no bacteria growth. Sample 24892-131E is a polyester fiber coated with anti-microbial polyester available commercially. This coated fiber showed trace bacteria growth. The results indicate that PP strips containing PAO/Ester/Triclosan showed anti-bacterial activity.

Table 4 below depicts the PAO/ester/Triclosan migration from the PP strips as measured by weight loss in 7 days.

TABLE 4
PAO/Ester/Triclosan Migration from PP Strips

| Run No. | Press at 180° C., Fast Cool (Quench at 7° C.) | | | | | |
|---|---|---|---|---|---|---|
| 24892-114 | 0 | 1 | 2 | 3 | 5 | 7 |
| PAO Type | PAO40 | PAO06 | PAO40 | PA0100 | Mix | Mix |
| | | | | | PAO40 | PAO030 |
| PP9355, | 85 | 85 | 85 | 85 | 85 | 85 |
| Wt % | | | | | | |
| Wt % | 15 | 14.25 | 14.25 | 14.25 | 14.25 | 14.25 |
| PAO/Ester | | | | | | |
| Wt % | 0 | 0.75 | 0.75 | 0.75 | 0.75 | 0.75 |
| Triclosan | | | | | | |
| % Wt loss | 1.04 | 1.58 | 1.78 | 0.84 | 0.88 | 1.50 |
| in 7 days | | | | | | |

For fast cooled PP strips, there was up to 1.8 wt % weight loss by toluene wash due to PAO/ester/Triclosan migration to the surface. The PAO, ester and triclosan all migrated to the surface as confirmed by GC analysis of the toluene wash.

Applicants have attempted to disclose all embodiments and applications of the disclosed subject matter that could be reasonably foreseen. However, there may be unforeseeable, insubstantial modifications that remain as equivalents. While the present invention has been described in conjunction with specific, exemplary embodiments thereof, it is evident that many alterations, modifications, and variations will be apparent to those skilled in the art in light of the foregoing description without departing from the spirit or scope of the present disclosure. Accordingly, the present disclosure is intended to embrace all such alterations, modifications, and variations of the above detailed description.

All patents, test procedures, and other documents cited herein, including priority documents, are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated.

What is claimed is:

1. A functional polymer composition comprising:
(a) at least 50 wt %, based on the total weight of the composition, of one or more thermoplastic polymers;
(b) from 0.1 to 50 wt %, based on the total weight of the composition, of one or more fluids;
(c) from 0.1 to 50 wt %, based on the total weight of the composition, of one or more active substrates; and
wherein an article formed from the composition exhibits one or more special functional effects from controlled release to the article surface of the one or more fluids and one or more active substrates.

2. The composition of claim 1, wherein the one or more special functional effects are selected from the group consisting of moisturizing, anti-bacterial, disinfecting, anti-viral, anti-mildew, anti-mold, anti-fungal, anti-microbial, moisture/odor absorbing, fragrancing, insect repelling, anti-static, and combinations thereof.

3. The composition of claim 1, wherein the one or more thermoplastic polymers are chosen from polyethylene, polypropylene, polybutene, EP copolymer, EB copolymer, PB copolymer, EPB terpolymer, EP copolymer with one or more C₄ to C₂₀ alpha-olefins, EVA copolymer, maleic anhydride modified polyethylene, maleic anhydride modified polypropylene, ethylene-alpha-olefin elastomers, polyolefin adhesive, styrene-polyethylene-alpha-olefin elastomers, ethylene elastomer, ethylene plastomer, styrene-diene copolymer, polyisobutylene rubber, PET, PBT, Nylon 6, Nylon 6,6, Nylon 4,6, Nylon 6,12, and combinations thereof.

4. The composition of claim 1 further including one or more additives chosen from natural fibers, fillers, cavitating agents, antioxidants, surfactants, adjuvants, plasticizers, block, antiblock, coloring agents, pigments, dyes, processing aids, UV stabilizers, neutralizers, waxes, nucleating agents, foaming agents, reinforcing fibers, antistatic agents, lubricating agents, clarifying agents and combinations thereof.

5. The composition of claim 1, wherein the one or more fluids are chosen from low molecular weight oligomers or polymers of olefins, ester fluids, polyester fluids, alkylated aromatic fluids, polyalkylene glycols, silicone fluids, polysiloxane fluids, hydrocarbon solvents, polyalpha-olefins, polyisobutylene, esters of mono-, di- or tri-basic acids, esters of acids with mono-, di- or polyols, EO/PO copolymers, polypropylene oxides, polybutyleneoxides, polytetrahydrofuran ether/ester/alcohol, alkylated naphthalene, alkylated aromatics, dimethicone, cyclomethicone, polychlorotrifluoroethylene, dialkyl bonates, polybutenes, paraffinic mineral oils, lubricant basestock derived from iso-paraffin-rich liquid derived from gas-to-liquid processes and combinations thereof.

6. The composition of claim 5, wherein the one or more fluids are a 4 to 1000 cS PAO fluid.

7. The composition of claim 5, wherein the ester fluids are chosen from maleates, adipates, azelates, sebacates, laureates, oleates, benzoates, phthalates, terephthalates, isophthalates, trimellitates, pyromellitate, dimerates, and esters of C₁ to C₂₂-aliphatic mono-acids.

8. The composition of claim 7 further including a PAO fluid, wherein the PAO fluid ranges from 1 to 50 wt %, based on the total weight of the fluids.

9. The composition of claim 1, wherein the one or more active substrates are chosen from petrolatum, aloe, PureSyn™ ester, dimethylcone, cyclomethicone, cetyl alcohol, stearyl alcohol, ceteary alcohol, PAO, mineral oil, squalane, squalene, microcrystalline wax, propylene glycol, glycerine, sorbitol, butylenes glycol, ethylene oxide-propylene oxide copolymer, PAG, PureSyn™ ester, isopropyl myristate, palmitate, state, octyl palmitate, jojoba, a mixture of nature esters vegetable oil, seed oil, other natural oils, PIB fluid, polyinternal olefin fluid and combinations thereof, and wherein the article formed exhibits a moisturizing effect.

10. The composition of claim 1, wherein the one or more active substrates are chosen from triclosan, triclocarban, antibacterial agents derived from quinolones, nitrofurans, sulfonamides, families of stable antibiotics and combinations thereof, and wherein the article formed exhibits an anti-bacterial effect.

11. The composition of claim 1, wherein the one or more active substrates are chosen from ethanol, isopropanol, butanol, zinc oxide, zinc halides, zinc acetate, zinc citrate, zinc gluconate, hexachlorophene, 4-hexylresorcinol, phenolic derivatives, hydroxybenzoic acids, hydroxybenzoic esters, salicylic acid, per-acids, quaternary ammonium compounds, surfactant disinfectants, peroxide compounds and combinations thereof, and wherein the article formed exhibits a disinfecting effect.

12. The composition of claim 1, wherein the one or more active substrates are chosen from amantadine, rimantadine, derivatives of amantadine, derivatives of rimantadine and combinations thereof, and wherein the article formed exhibits an anti-viral effect.

13. The composition of claim 1, wherein the one or more active substrates are chosen from p-dichlorobenzene, paraformaldehyde, wax resin, silicone resin, salicyanilide in IPA, oxalic acid, potassium sorbated, calcium sorbated, sorbic acid, boric acid and its derivatives, sodium pyrithione, zinc pyrithione, amantadine and combinations thereof, wherein the article formed exhibits an anti-mildew effect.

14. The composition of claim 1, wherein the one or more active substrates are chosen from allylamine, azoles, polyenes, ciclopirox olamine, undecylenate and combinations thereof, and wherein the article formed exhibits an anti-fungal effect.

15. The composition of claim 1, wherein the one or more active substrates are chosen from aluminum chlorohydrate, aluminum zirconium chloride hydroxide complex, activated aluminum chlorohydrate and aluminum zirconium chloride hydroxide complex, zinc phenolsulfonate, p-chloro-m-xylenol and combinations thereof, and wherein the article formed exhibits a moisture and odor absorbing effect.

16. The composition of claim 1, wherein the one or more active substrates are chosen from citronellene, citronellol, citronellal, citral, linalool, menthol, (R)-(+)-β-citronellol, (S)-(+)-β-citronellol, geraniol, ester derivatives of the preceding compounds, jasmone, terpenes, derivatives of jasmone, derivatives of terpenes, 2-phenylethanol and its derivatives, benzaldehyde and its derivatives and combinations thereof, and wherein the article formed exhibits a fragrance effect.

17. The composition of claim 1, wherein the one or more active are chosen from N,N-diethyl-m-toluamide (DEET), dimethyl phthalate, 2-ethyl-1,3-hexadiol, indalone and combinations thereof, wherein the article formed exhibits an insect repellant effect.

18. The composition of claim 1, wherein the one or more active substrates are chosen from long-chain amines, amides and quaternary ammonium salts; esters of fatty acids and their derivatives, sulfonic acids, alkyl aryl sulfonates; polyoxyethylene derivatives; polyalkylene glycols and their derivatives, polyhydric alcohols and their derivatives, phosphoric acid derivatives, molten salts, water, combinations thereof, wherein the article formed exhibits an antistatic effect.

19. The composition of claim 1 further including EDTA as a promoting agent to provide for controlled or delayed release of the one or more fluids and the one or more active substrates to the article surface.

20. The composition of claim 1, wherein the article is selected from the group consisting of a film, a molded article, a sheet, a fiber, a melt blown fabric, a spun bonded fabric, a non-woven fabric, a woven fabric, an adhesive, and a wax.

21. The composition of claim 1, wherein the one or more fluids and/or one or more active substrates are partially or wholly absorbed inside a porous, mesoporous or microporous material.

22. The composition of claim 21, wherein the porous material is chosen from porous silica, alumina, silicoaluminate, zirconia, and titania.

23. The composition of claim 21, wherein the mesoporous material is chosen from MCM41, SBA-15, FSM-16, zeolites, MCM22, MCM49, MCM56, MCM36, natural clays, synthetic clays and combinations thereof.

24. The composition of claim 23, wherein the mesoporous material comprises MCM41, SBA-15 and FSM-16.

25. The composition of claim 23, wherein the mesoporous material comprises zeolites, MCM22, MCM49, MCM56, MCM36, natural clays and synthetic clays.

26. A functional polymer composition comprising:
(a) at least 50 wt %, based on the total weight of the composition, of one or more thermoplastic polymers; and
(b) from 0.1 to 50 wt %, based on the total weight of the composition, of one or more fluids;
wherein an article formed from the composition exhibits one or more special functional effects from controlled-release to the article surface of the one or more fluids.

27. The composition of claim 26, wherein the one or more thermoplastic polymers are chosen from polyethylene, polypropylene, polybutene, EP copolymer, EB copolymer, PB copolymer, EPB terpolymer, EP copolymer with one or more C₄ to C₂₀ alpha-olefins, EVA copolymer, maleic anhydride modified polyethylene, maleic anhydride modified polypropylene, ethylene-alpha-olefin elastomers, polyolefin adhesive, styrene-polyethylene-alpha-olefin elastomers, ethylene elastomer, ethylene plastomer, styrene-diene copolymer, polyisobutylene rubber, PET, PBT, Nylon 6, Nylon 6,6, Nylon 4,6, Nylon 6,12, and combinations thereof.

28. The composition of claim 26, wherein the one or more fluids are chosen from low molecular weight oligomers or polymers of olefins, ester fluids, polyester fluids, alkylated aromatic fluids, polyalkylene glycols, silicone fluids, polysiloxane fluids, hydrocarbon solvents, polyalpha-olefins, polyisobutylene, esters of mono-, di- or tri-basic acids, esters of acids with mono-, di- or polyols, EO/PO copolymers, polypropylene oxides, polybutyleneoxides, polytetrahydrofuran ether/ester/alcohol, alkylated naphthalene, alkylated aromatics, dimethicone, cyclomethicone, polychlorotrifluoroethylene, dialkyl bonates, polybutenes, paraffinic mineral oils, lubricant basestock derived from iso-paraffin-rich liquid derived from gas-to-liquid processes and combinations thereof, and wherein the article formed exhibits a moisturizing effect.

29. The composition of claim 26, wherein the one or more fluids are a 4 to 1000 cS PAO fluid.

30. The composition of claim 26 further including one or more additives chosen from natural fibers, fillers, cavitating agents, antioxidants, surfactants, adjuvants, plasticizers, block, antiblock, coloring agents, pigments, dyes, processing aids, UV stabilizers, neutralizers, waxes, nucleating agents, foaming agents, reinforcing fibers, antistatic agents, lubricating agents, clarifying agents and combinations thereof.

31. The composition of claim 26 further including EDTA as a promoting agent to provide for controlled or delayed release of the one or more fluids to the article surface.

32. The composition of claim 26, wherein the article is selected from the group consisting of a film, a molded article, a sheet, a fiber, a melt blown fabric, a spun bonded fabric, a non-woven fabric, a woven fabric, an adhesive, and a wax.

33. The composition of claim 26, wherein the one or more fluids are partially or wholly absorbed inside a porous, mesoporous or microporous material.

34. The composition of claim 33, wherein the mesoporous material is chosen from MCM41, SBA-15, FSM-16, zeolites, MCM22, MCM49, MCM56, MCM36, natural clays, and synthetic clays.

35. The composition of claim 33, wherein the porous material is chosen from porous silica, alumina, silicoaluminate, zirconia, and titania.

36. A functional polymer composition comprising:
(a) at least 50 wt %, based on the total weight of the composition, of one or more thermoplastic polymers;
(b) from 0.1 to 50 wt %, based on the total weight of the composition, of one or more active substrates; and
wherein an article formed from the composition exhibits one or more special functional effects from controlled release of the one or more active substrates to the article surface.

37. The composition of claim 36, wherein the one or more special functional effects are selected from the group consisting of moisturizing, anti-bacterial, disinfecting, anti-viral, anti-mildew, anti-mold, anti-fungal, anti-microbial, moisture/odor absorbing, fragrancing, insect repelling, anti-static and combinations thereof.

38. The composition of claim 36, wherein the one or more thermoplastic polymers are chosen from polyethylene, polypropylene, polybutene, EP copolymer, EB copolymer, PB copolymer, EPB terpolymer, EP copolymer with one or more C₄ to C₂₀ alpha-olefins, EVA copolymer, maleic anhydride modified polyethylene, maleic anhydride modified polypropylene, ethylene-alpha-olefin elastomers, polyolefin adhesive, styrene-polyethylene-alpha-olefin elastomers, ethylene elastomer, ethylene plastomer, styrene-diene copolymer, polyisobutylene rubber, PET, PBT, Nylon 6, Nylon 6,6, Nylon 4,6, Nylon 6,12, and combinations thereof.

39. The composition of claim 36 further including one or more additives chosen from natural fibers, fillers, cavitating agents, antioxidants, surfactants, adjuvants, plasticizers, block, antiblock, coloring agents, pigments, dyes, processing aids, UV stabilizers, neutralizers, waxes, nucleating agents, foaming agents, reinforcing fibers, antistatic agents, lubricating agents, clarifying agents and combinations thereof.

40. The composition of claim 36, wherein the one or more active agents are chosen from petrolatum, aloe, PureSyn™ ester, dimethylcone, cyclomethicone, cetyl alcohol, stearyl alcohol, ceteary alcohol, PAO, mineral oil, squalane, squalene, microcrystalline wax, propylene glycol, glycerine, sorbitol, butylenes glycol, ethylene oxide-propylene oxide copolymer, PAG, PureSyn™ ester, isopropyl myristate, palmitate, state, octyl palmitate, jojoba, a mixture of nature esters vegetable oil, seed oil, other natural oils, PIB fluid, polyinternal olefin fluid and combinations thereof, and wherein the article formed exhibits a moisturizing effect.

41. The composition of claim 36, wherein the one or more active substrates are chosen from triclosan, triclocarban, antibacterial agents derived from quinolones, nitrofurans, sulfonamides, families of stable antibiotics and combinations thereof, and wherein the article formed exhibits an anti-bacterial effect.

42. The composition of claim 36, wherein the one or more active substrates are chosen from ethanol, isopropanol, butanol, zinc oxide, zinc halides, zinc acetate, zinc citrate, zinc gluconate, hexachlorophene, 4-hexylresorcinol, phenolic derivatives, hydroxybenzoic acids, hydroxybenzoic esters, salicylic acid, per-acids, quaternary ammonium compounds, amphoteric surfactant disinfectants, peroxide compounds and combinations thereof, and wherein the article formed exhibits a disinfecting effect.

43. The composition of claim 36, wherein the one or more active substrates are chosen from amantadine, rimantadine, derivatives of amantadine, derivatives of rimantadine and combinations thereof, and wherein the article formed exhibits an anti-viral effect.

44. The composition of claim 36, wherein the one or more active substrates are chosen from p-dichlorobenzene, paraformaldehyde, wax resin, silicone resin, salicyanilide in IPA, oxalic acid, potassium sorbated, calcium sorbated, sorbic acid, boric acid and its derivatives, sodium pyrithione, zinc pyrithione, amantadine and combinations thereof, wherein the article formed exhibits an anti-mildew effect.

45. The composition of claim 36, wherein the one or more active substrates are chosen from allylamine, azoles, polyenes, ciclopirox olamine, undecylenate and combinations thereof, and wherein the article formed exhibits an anti-fungal effect.

46. The composition of claim 36, wherein the one or more active substrates are chosen from aluminum chlorohydrate, aluminum zirconium chloride hydroxide complex, activated aluminum chlorohydrate and aluminum zirconium chloride hydroxide complex, zinc phenolsulfonate, p-chloro-m-xylenol and combinations thereof, and wherein the article formed exhibits a moisture and odor absorbing effect.

47. The composition of claim 36, wherein the one or more active substrates are chosen from citronellene, citronellol, citronellal, citral, linalool, menthol, (R)-(+)-β-citronellol, (S)-(+)-β-citronellol, geraniol, ester derivatives of the preceding compounds, jasmone, terpenes, derivatives of jasmone, derivatives of terpenes, 2-phenylethanol and its derivatives, benzaldehyde and its derivatives and combinations thereof, and wherein the article formed exhibits a fragrance effect.

48. The composition of claim 36, wherein the one or more active substrates are chosen from N,N-diethyl-m-toluamide (DEET), dimethyl phthalate, 2-ethyl-1,3-hexadiol, indalone and combinations thereof, wherein the article formed exhibits an insect repellant effect.

49. The composition of claim 36, wherein the one or more active substrates are chosen from long-chain amines, amides and quaternary ammonium salts; esters of fatty acids and their derivatives, sulfonic acids, alkyl aryl sulfonates; polyoxyethylene derivatives; polyalkylene glycols and their derivatives, polyhydric alcohols and their derivatives, phosphoric acid derivatives, molten salts, water, combinations thereof, wherein the article formed exhibits an antistatic effect.

50. The composition of claim 36 further including EDTA as a promoting agent to provide for controlled or delayed release of the one or more active substrates to the article surface.

51. The composition of claim 36, wherein the article is selected from the group consisting of a film, a molded article, a sheet, a fiber, a melt blown fabric, a spun bonded fabric, a non-woven fabric, a woven fabric, an adhesive, and a wax.

52. The composition of claim 36, wherein the one or more active agents are partially or wholly absorbed inside a porous, mesoporous or microporous material.

53. The composition of claim 36, wherein the mesoporous material is chosen from MCM41, SBA-15, FSM-16, zeolites, MCM22, MCM49, MCM56, MCM36, natural clays, and synthetic clays.

54. The composition of claim 36, wherein the porous material is chosen from porous silica, alumina, silicoaluminate, zirconia, and titania.

55. A functional polymer composition comprising:
(a) at least 50 wt %, based on the total weight of the composition, of one or more thermoplastic polymers;
(b) from 0.1 to 50 wt %, based on the total weight of the composition, of a fluid mixture of PAO and ester fluids,
wherein the fluid mixture comprises 50 to 99 wt % of PAO fluid and 1 to 50 wt % of ester fluid, based on the total weight of the fluid mixture;
(c) from 0.1 to 30 wt %, based on the total weight of the composition, of triclosan; and
wherein an article formed from the composition exhibits an anti-bacterial effect from migration to the article surface of the fluid mixture and the triclosan.

56. The composition of claim 55, wherein the one or more thermoplastic polymers are chosen from polyethylene, polypropylene, polybutene, EP copolymer, EB copolymer, PB copolymer, EPB terpolymer, EP copolymer with one or more C₄ to C₂₀ alpha-olefins, EVA copolymer, maleic anhydride modified polyethylene, maleic anhydride modified polypropylene, ethylene-alpha-olefin elastomers, polyolefin adhesive, styrene-polyethylene-alpha-olefin elastomers, ethylene elastomer, ethylene plastomer, styrene-diene copolymer, polyisobutylene rubber, PET, PBT, Nylon 6, Nylon 6,6, Nylon 4,6, Nylon 6, 12, and combinations thereof.

57. The composition of claim 56, wherein the one or more thermoplastic polymers is polypropylene.

58. The composition of claim 57, wherein the ester fluid is di-tridecyl adipate or di-tridecyl phthalate.

59. The composition of claim 58, wherein the anti-bacterial effect includes no anti-bacterial activity towards staphylococcus aureus and klebiella pneumoniae.

60. The composition of claim 53, wherein the article is selected from the group consisting of a film, a molded article, a sheet, a fiber, a melt blown fabric, a spun bonded fabric, a non-woven fabric, a woven fabric, an adhesive, and a wax.

61. The composition of claim 55, wherein the fluid mixture of PAO and ester fluids are partially or wholly absorbed inside a porous, mesoporous or microporous material.

62. The composition of claim 61, wherein the mesoporous material is chosen from MCM41, SBA-15, FSM-16, zeolites, MCM22, MCM49, MCM56, MCM36, natural clays, and synthetic clays.

63. The composition of claim 61, wherein the porous material is chosen from porous silica, alumina, silicoaluminate, zirconia, and titania.

64. A method of forming a functional polymer composition comprising:
providing a composition comprising:
(a) at least 50 wt %, based on the total weight of the composition, of one or more thermoplastic polymers;
(b) from 0.0 to 50 wt %, based on the total weight of the composition, of one or more fluids;
(c) from 0.1 to 50 wt %, based on the total weight of the composition, of one or more active substrates agents; and
mixing the composition, and
forming the composition into an article,
wherein the article exhibits one or more special functional effects from controlled release to the article surface of the one or more fluids and one or more active substrates.

65. The method of claim 64, wherein the mixing step is done in a single screw compounding extruder, a twin screw compounding extruder, or a batch type mixer.

66. The method of claim 64, wherein the one or more special functional effects are selected from the group consisting of moisturizing, anti-bacterial, disinfecting, anti-viral, anti-mildew, anti-mold, anti-fungal, anti-microbial, moisture/odor absorbing, fragrancing, insect repelling, anti-static and combinations thereof.

67. The method of claim 64, wherein the forming step is selected from the group consisting of extruding, molding, thermoforming, fiber spinning, melt blowing, spunbonding, and fiber weaving.

68. The method of claim 64, wherein the article is selected from the group consisting of a film, a molded article, a sheet, a fiber, a melt blown fabric, a spun bonded fabric, a non-woven fabric, a woven fabric, an adhesive, and a wax.

69. The method of claim 64, wherein the one or more fluids and/or one or more active substrates are partially or wholly absorbed inside a porous, mesoporous or microporous material.

70. The composition of claim 69, wherein the mesoporous material is chosen from MCM41, SBA-15, FSM-16, zeolites, MCM22, MCM49, MCM56, MCM36, natural clays, and synthetic clays.

71. The composition of claim 69, wherein the porous material is chosen from porous silica, alumina, silicoaluminate, zirconia, and titania.

72. A method of forming a functional polymer based article comprising:
providing a fluid composition comprising:
(a) from 0.0 to 95 wt %, based on the total weight of the composition, of one or more fluids;
(b) from 0.1 to 95 wt %, based on the total weight of the composition, of one or more active substrates; and
mixing the fluid composition; and
spraying or impregnating the fluid composition onto the surface of a polymer based article,
wherein the article exhibits one or more special functional effects from migration to the article surface of the one or more fluids and one or more active substrates.

73. The method of claim 72, wherein the functional polymer composition provides one or more special functional effects selected from the group consisting of moisturizing, anti-bacterial, disinfecting, anti-viral, anti-mildew, anti-mold, anti-fungal, anti-microbial, moisture/odor absorbing, fragrancing, insect repelling, anti-static and combinations thereof.

74. The method of claim 72, wherein the polymer based article is selected from the group consisting of a film, a molded article, a sheet, a fiber, a melt blown fabric, a spunbonded fabric, a non-woven fabric, a woven fabric, an adhesive, and a wax.

75. The method of claim 72, wherein the one or more fluids and/or one or more active substrates are partially or wholly absorbed inside a porous, mesoporous or microporous material.

76. The composition of claim 72, wherein the mesoporous material is chosen from MCM41, SBA-15, FSM-16, zeolites, MCM22, MCM49, MCM56, MCM36, natural clays, and synthetic clays.

77. The composition of claim 72, wherein the porous material is chosen from porous silica, alumina, silicoaluminate, zirconia, and titania.
